(19) United States
(12) Patent Application Publication
(10) Pub. No.: US 2021/0047693 A1
(43) Pub. Date: Feb. 18, 2021

(54) METHODS OF TREATING EXTRACHROMOSOMAL DNA EXPRESSING CANCERS

(71) Applicants: THE REGENTS OF THE UNIVERSITY OF CALIFORNIA, Oakland, CA (US); LUDWIG INSTITUTE FOR CANCER RESEARCH LTD, Zurich (CH)

(72) Inventors: Paul MISCHEL, Zurich (CH); Vineet BAFNA, Oakland, CA (US); Junho KO, Zurich (CH); Wenjing ZHANG, Zurich (CH); Utkrisht RAJKUMAR, Oakland, CA (US)

(21) Appl. No.: 16/764,569

(22) PCT Filed: Nov. 15, 2018
(86) PCT No.: PCT/US2018/061376

§ 371 Date: May 15, 2020

Related U.S. Application Data
(60) Provisional application No. 62/586,731, filed on Nov. 15, 2017.

(51) Int. Cl. C12Q 1/6886; A61K 31/55; A61K 31/502; A61P 35/00

(52) U.S. Cl. CPC C12Q 1/6886; A61K 31/55; C12Q 2600/106; A61P 35/00; C12Q 2600/156; A61K 31/502

(57) ABSTRACT

Provided herein are, inter alia, methods of treating cancer in a subject having or being at risk of developing cancer, wherein the subject has an amplified extrachromosomal oncogene. The treatment methods provided herein target cancer cells that include extrachromosomal DNA by administering a therapeutically effective amount of a DNA repair pathway inhibitor (e.g., a PARP inhibitor). The methods provided herein are furthermore useful to indicate the progressiveness of cancer, and/or to facilitate evaluation of responsiveness to therapy.

### CROSS-REFERENCES TO RELATED APPLICATIONS

This application claims the benefit of U.S. Provisional Application No. 62/586,731, filed Nov. 15, 2017, which is incorporated herein by reference in entirety and for all purposes.

### STATEMENT AS TO RIGHTS TO INVENTIONS MADE UNDER FEDERALLY SPONSORED RESEARCH AND DEVELOPMENT

This invention was made with government support under grant number GM114362 awarded by the National Institutes of Health. The government has certain rights in the invention.

### BACKGROUND

Human cells have twenty-three pairs of chromosomes but in cancer, genes can be amplified in chromosomes or in circular extrachromosomal DNA (ECDNA), whose frequency and functional significance are not understood¹⁻⁴. We performed whole genome sequencing, structural modeling and cytogenetic analyses of 17 different cancer types, including 2572 metaphases, and developed ECdetect to conduct unbiased integrated ECDNA detection and analysis. ECDNA was found in nearly half of human cancers varying by tumor type, but almost never in normal cells. Driver oncogenes were amplified most commonly on ECDNA, elevating transcript level. Mathematical modeling predicted that ECDNA amplification elevates oncogene copy number and increases intratumoral heterogeneity more effectively than chromosomal amplification, which we validated by quantitative analyses of cancer samples. These results suggest that ECDNA contributes to accelerated evolution in cancer.

Cancers evolve in rapidly changing environments from single cells into genetically heterogeneous masses. Darwinian evolution selects for those cells better fit to their environment. Heterogeneity provides a pool of mutations upon which selection can act^{1,5-9}. Cells that acquire fitness-enhancing mutations are more likely to pass these mutations on to daughter cells, driving neoplastic progression and therapeutic resistance^{10,11}. One common type of cancer mutation, oncogene amplification, can be found either in chromosomes or nuclear ECDNA elements, including double minutes (DMs)^{2-4,12-14}. Relative to chromosomal amplicons, ECDNA is less stable, segregating unequally to daughter cells^{15,16}. DMs are reported to occur in 1.4% of cancers with a maximum of 31.7% in neuroblastoma, based on the Mitelman database^{4,7}. However, the scope of ECDNA in cancer has not been accurately quantified, the oncogenes contained therein have not been systematically examined, and the impact of ECDNA on tumor evolution has yet to be determined.

There is a need in the art for the targeted treatment of ecDNA cancers and personalized treatment methods that make use of the differential expression of extrachromosomal DNA in cancer cell. The methods and compositions provided herein, inter alia, address these and other needs in the art.

### BRIEF SUMMARY OF THE INVENTION

In one aspect, a method of treating cancer in a human subject having or being at risk of developing cancer is provided. The method includes administering to the human subject an effective amount of a DNA repair pathway inhibitor, thereby treating cancer in the subject, wherein the human subject has an amplified extrachromosomal oncogene.

In one aspect, a method of treating cancer in a human subject having or being at risk of developing cancer is provided. The method includes administering to the human subject an effective amount of a DNA repair pathway inhibitor, thereby treating cancer in the subject, wherein the human subject has been identified as having an amplified extrachromosomal oncogene.

In one aspect, a method of treating cancer in a human subject in need thereof is provided. The method includes (i) detecting an amplified extrachromosomal oncogene in a cancer cell in a first biological sample obtained from a human subject having or being at risk of developing cancer by contacting the biological sample with an oncogene-binding agent and detecting binding of the oncogene-binding agent to the amplified extrachromosomal oncogene; and (ii) administering to the human subject an effective amount of a DNA repair pathway inhibitor thereby treating cancer in the subject.

In one aspect, a method of treating cancer in a human subject in need thereof is provided. The method includes (i) detecting a first level of an amplified extrachromosomal oncogene in a cancer cell in a first biological sample obtained from a human subject having or being at risk of developing cancer; (ii) administering to the human subject an effective amount of a DNA repair pathway inhibitor; (iii) detecting a second level of an amplified extrachromosomal oncogene in a cancer cell in a second biological sample obtained from the human subject; and (iv) comparing the first level to the second level, thereby treating cancer in the human subject.

### BRIEF DESCRIPTION OF THE DRAWINGS

FIGS. 1A-1C. The figures show that the EGFR inhibitor erlotinib causes the formation of EGFR+micronuclei. FIG. 1A shows measurement by visualization of interphase cells stained with an EGFR FISH probe. FIG. 1B shows visualization of EGFR and CEN7. FIG. 1C shows measurement by physical purification of micronuclei by centrifugation, followed by visualization with an EGFR FISH probe.

FIGS. 2A-2B. The figures show that the EGFR inhibitor erlotinib causes the loss of ecDNA containing amplified EGFRvIII. FIG. 2A shows number of ecDNAs per metaphase. FIG. 2B shows visualization of EGFR and CEN7.

FIG. 3 The figure shows that other EGFR tyrosine kinase inhibitors similarly cause the formation of EGFR-containing micronuclei and cause loss of EGFR-containing ecDNA in GBM cells—findings have been confirmed in multiple patient-derived GBM neurosphere cultures.

FIGS. 4A-4B. The figures show reduction of cellular level of oncogenes amplified on ecDNA in response to targeted inhibitor treatment via exosomal export. FIG. 4A shows FISH probe-based analysis of exosomes purified from GBM39 cells (Mol Cancer Ther. 2007 March; 6(3):1167-74) treated with erlotinib. FIG. 4B shows PCR analysis of exosomes purified from GBM39 cells treated with erlotinib.

FIGS. 5A-5B. The figures show that the addition of deoxy-nucleotides prevents DNA damage on extrachromosomal DNA in response to targeted inhibitors, which does not occur on chromosomal DNA, and prevents formation of micronuclei from oncogenes amplified on ecDNA. FIG. 5A shows the frequency of rH2AX*ecDNA. FIG. 5B shows the number of micronuclei from 500 primary nucleus.

FIGS. 6A-6B. The figures show that glucose withdrawal causes the formation of EGFR+micronuclei in GBM cells similar to erlotinib. Erlotinib treatment lowers glucose levels in GBM cells indicating that the effects of erlotinib on mincronuclei are mediated through the control of glucose update and utilization. FIG. 6A shows the number of micronuclei from 500 primary nucleus. FIG. 6B shows glucose (g/l/10\hat{ }6 cells).

FIGS. 7A-7B. The figures show that glucose withdrawal causes the formation of micronuclei containing the oncogene amplified on ecDNA. In GBM cells, erlotinib treatment or glucose withdrawal similarly induce EGFR+micronuclei formation, both of which are rescued by adding deoxy-ribonucleotides. These data demonstrate a unique dependence of ecDNA on de novo nucleotide synthesis from glucose, which is driven by the oncogenes amplified on ecDNA. FIG. 7A shows the number of micronuclei from 500 primary nucleus. FIG. 7B shows the number of EGFR+ micronuclei.

FIG. 8. The figure shows that glucose withdrawal specifically damages ecDNA.

FIG. 9. The figure shows that dependence of ecDNA on glucose for de novo nucleotide is seen across a range of cancers with a spectrum of amplified oncogenes including prostate cancer with c-Myc amplification.

FIGS. 10A-10B. The figures show that the ability of ecDNA to replicate is specifically suppressed by glucose withdrawal in glioblastoma and prostate cancer cells. The replication kinetics of chromosomal DNA remains unaffected, highlighting the unique metabolic vulnerability of ecDNA. FIG. 10A shows GBM39 ecDNA subclone cells. FIG. 10B shows PC3 cells.

FIGS. 11A-11B. The figures show that erlotinib treatment specifically causes replication stress on ecDNA, but not on chromosomal DNA. FIG. 11A shows frequency of p333 on ecDNA. FIG. 11B shows frequency of pRPA(533) positive metaphase for vehicle versus erlotinib.

FIGS. 12A-12C. Cells containing ecDNA are sensitive to PARP inhibition. FIG. 12A) Acute cell toxicity following 4 days treatment with 10 μM of indicated PARPi. Cell death measured by FACS analysis of Sytox Red staining in 2 normal cell types (astrocytes and HEK293), PC3 ecDNA-containing cells, and the paired GBM39 cells. FIG. 12B) 2D colony formation assay and crystal violet staining in immortalized HEK293 cells and PC3 cells after treatment with Olaparib or Rucaparib. FIG. 12C) Colony number quantification by Colony Area software plug-in for ImageJ from data in (FIG. 12B).

FIGS. 13A-13B. Cells containing ecDNA are sensitive to PARP inhibition. FIG. 13A) 3D soft agar assay in isogenic paired GBM39 cells treated with Olaparib or Rucaparib. Quantification of colonies as measured by ColonyArea software (bottom). FIG. 13B) 3D soft agar assay in isogenic paired COLO320 cells (a colon cancer cell line) treated with Olaparib or Rucaparib. Quantification of colonies as measured by ColonyArea software (bottom).

FIGS. 14A-14C. Decreased number of ecDNA in GBM39 cells cultured in low glucose: GBM39 cells were maintained in medium with low glucose (3.5 mM) or normal glucose (17.5 mM) respectively for 4 weeks. Metaphase spreads were stained with DAPI, and ecDNA numbers were analyzed with ecDetect. More than 50 metaphase cells were analyzed in each group. FIG. 14A. Representative image of original image and ecDNAs showed by ecDetect. FIG. 14B. Histogram distribution graph of ecDNA numbers per cell in each group. FIG. 14C. Quantification analysis of average number of ecDNAs per cell.

FIGS. 15A-15E. Decreased number of ecDNAs and EGFR copy in GBM39 cells cultured in low glucose: GBM39 cells were maintained in medium with low glucose (3.5 mM) or normal glucose (17.5 mM) for 4 weeks. FISH probe with EGFR was stained in metaphase spreads with co-staining with DAPI, and both ecDNA numbers (DAPI signal) and EGFR copy number (EGFR signal) were analyzed with ecDetect. More than 50 metaphase cells were analyzed in each group. FIG. 15A. Representative image. FIG. 15B. Histogram distribution graph of ecDNA numbers per cell in each group. FIG. 15C. Quantification analysis of average number of ecDNAs per cell. FIG. 15D. Histogram distribution graph of EGFR copy number per cell in each group. FIG. 15E. Quantification analysis of average number of EGFR copy number per cell.

FIGS. 16A-16C. Decreased number of ecDNA in HK359 cells cultured in low glucose: HK359 cells were maintained in medium with low glucose (3.5 mM) or normal glucose (17.5 mM) respectively for 4 weeks. Metaphase spreads were stained with DAPI, and ecDNA numbers were analyzed with ecDetect. More than 50 metaphase cells were analyzed in each group. FIG. 16A. Representative image of original image and ecDNAs showed by ecDetect. FIG. 16B. Histogram distribution graph of ecDNA numbers per cell in each group. FIG. 16C. Quantification analysis of average number of ecDNAs per cell.

FIGS. 17A-17E. Decreased number of ecDNAs and EGFR copy in HK359 cells cultured in low glucose: HK359 cells were maintained in medium with low glucose (3.5 mM) or normal glucose (17.5 mM) for 4 weeks. FISH probe with EGFR was stained in metaphase spreads with co-staining with DAPI, and both ecDNA numbers (DAPI signal) and EGFR copy number (EGFR signal) were analyzed with ecDetect. More than 50 metaphase cells were analyzed in each group. FIG. 17A. Representative image. FIG. 17B. Histogram distribution graph of ecDNA numbers per cell in each group. FIG. 17C. Quantification analysis of average number of ecDNAs per cell. FIG. 17D. Histogram distribution graph of EGFR copy number per cell in each group. FIG. 17E. Quantification analysis of average number of EGFR copy number per cell.

FIGS. 18A-18C. Decreased number of ecDNA in PC3 cells cultured in low glucose: PC3 cells were maintained in medium with low glucose (5 mM) or normal glucose (25 mM) for 4 weeks. Metaphase spreads were stained with DAPI, and ecDNA numbers were counted. More than 50 metaphase cells were analyzed in each group. FIG. 18A. Representative image. FIG. 18B. Histogram distribution graph of ecDNA numbers per cell in each group. FIG. 18C. Quantification analysis of average number of ecDNAs per cell.

FIGS. 19A-19B. Decreased number of myc copy number in PC3 cells cultured in low glucose: PC3 cells were maintained in medium with low glucose (5 mM) or normal glucose (25 mM) for 4 weeks. Metaphase spreads were stained with myc FISH probe with co-staining with DAPI, and myc copy number in each cell were counted. More than 50 metaphase cells were analyzed in each group. FIG. 19A. Histogram distribution graph of myc copy numbers per cell in each group. FIG. 19B. Quantification analysis of average myc copy numbers per cell.

FIGS. 20A-20C. Decreased number of ecDNA in Colo320-DM cells cultured in low glucose: Colo320-DM cells were maintained in medium with low glucose (5 mM) or normal glucose (25 mM) for 4 weeks. Metaphase spreads were stained with DAPI, and ecDNA numbers were counted. More than 50 metaphase cells were analyzed in each group. FIG. 20A. Representative image. FIG. 20B. Histogram distribution graph of ecDNA numbers per cell in each group. FIG. 20C. Quantification analysis of average number of ecDNAs per cell.

FIGS. 21A-21B. Decreased number of myc copy number in Colo320-DM cells cultured in low glucose: Colo320-DM cells were maintained in medium with low glucose (5 mM) or normal glucose (25 mM) for 4 weeks. Metaphase spreads were stained with myc FISH probe with co-staining with DAPI, and myc copy number in each cell were counted. More than 50 metaphase cells were analyzed in each group. FIG. 21A. Histogram distribution graph of myc copy numbers per cell in each group. FIG. 21B. Quantification analysis of average myc copy numbers per cell.

FIG. 22. Increased engulfment of ecDNAs into micronuclei in GBM39 cells maintained with low glucose. GBM39 cells were maintained in low glucose (3.5 mM) or normal glucose (17.5 mM) for 4 weeks. Interphase cells were collected and stained with EGFR FISH probe. Micronuclei numbers and EGFR positive micronuclei numbers were counted in the number of cells indicated.

FIG. 23. Increased engulfment of ecDNAs into micronuclei in HK359 cells maintained with low glucose. HK359 cells were maintained in low glucose (3.5 mM) or normal glucose (17.5 mM) for 4 weeks. Interphase cells were collected and stained with EGFR FISH probe. Micronuclei numbers and EGFR positive micronuclei numbers were counted in the number of cells indicated.

### DETAILED DESCRIPTION

I. Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

The term “small molecule” as used herein refers to a low molecular weight organic compound that may regulate a biological process. In embodiments, small molecules are drugs. In embodiments, small molecules have a molecular weight less than 900 daltons. In embodiments, small molecules are of a size on the order of one nanometer.

The term “organic compound” as used herein refers to any of a large class of chemical compounds in which one or more atoms of carbon are covalently linked to atoms of other elements.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers to nucleic acids that have the same basic chemical structure as a naturally occurring nucleic acid. Such analogues have modified sugars and/or modified ring substituents, but retain the same basic chemical structure as the naturally occurring nucleic acid. A nucleic acid mimetic refers to chemical compounds that have a structure that is different from the general chemical structure of a nucleic acid, but that functions in a manner similar to a naturally occurring nucleic acid. Examples of such analogues include, without limitation, phosphorothiolates, phosphoramidates, methyl phosphonates, chiral-methyl phosphonates, 2-O-methyl ribonucleotides, and peptide-nucleic acids (PNAs).

Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

An “antisense nucleic acid” as referred to herein is a nucleic acid (e.g., DNA or RNA molecule) that is complementary to at least a portion of a specific target nucleic acid and is capable of reducing transcription of the target nucleic acid (e.g. mRNA from DNA), reducing the translation of the target nucleic acid (e.g. mRNA), altering transcript splicing (e.g. single stranded morpholino oligo), or interfering with the endogenous activity of the target nucleic acid. See, e.g., Weintraub, Scientific American, 262:40 (1990). Typically, synthetic antisense nucleic acids (e.g. oligonucleotides) are generally between 15 and 25 bases in length. Thus, antisense nucleic acids are capable of hybridizing to (e.g. selectively hybridizing to) a target nucleic acid. In embodiments, the antisense nucleic acid hybridizes to the target nucleic acid in vitro. In embodiments, the antisense nucleic acid hybridizes to the target nucleic acid in a cell. In embodiments, the antisense nucleic acid hybridizes to the target nucleic acid in an organism. In embodiments, the antisense nucleic acid hybridizes to the target nucleic acid under physiological conditions. Antisense nucleic acids may comprise naturally occurring nucleotides or modified nucleotides such as, e.g., phosphorothioate, methylphosphonate, and -anomeric sugar-phosphate, backbone modified nucleotides.

In the cell, the antisense nucleic acids hybridize to the corresponding RNA forming a double-stranded molecule. The antisense nucleic acids interfere with the endogenous behavior of the RNA and inhibit its function relative to the absence of the antisense nucleic acid. Furthermore, the double-stranded molecule may be degraded via the RNAi pathway. The use of antisense methods to inhibit the in vitro translation of genes is well known in the art (Marcus-Sakura, Anal. Biochem., 172:289, (1988)). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

As used herein, the term “vector” refers to a nucleic acid molecule capable of transporting another nucleic acid to which it has been linked. One type of vector is a “plasmid”, which refers to a linear or circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. Additionally, some viral vectors are capable of targeting a particular cells type either specifically or non-specifically. Replication-incompetent viral vectors or replication-defective viral vectors refer to viral vectors that are capable of infecting their target cells and delivering their viral payload, but then fail to continue the typical lytic pathway that leads to cell lysis and death.

The terms “transfection”, “transduction”, “transfecting” or “transducing” can be used interchangeably and are defined as a process of introducing a nucleic acid molecule and/or a protein to a cell. Nucleic acids may be introduced to a cell using non-viral or viral-based methods. The nucleic acid molecule can be a sequence encoding complete proteins or functional portions thereof. Typically, a nucleic acid vector, comprising the elements necessary for protein expression (e.g., a promoter, transcription start site, etc.). Non-viral methods of transfection include any appropriate method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. For viral-based methods, any useful viral vector can be used in the methods described herein. Examples of viral vectors include, but are not limited to retroviral, adenoviral, lentiviral and adeno-associated viral vectors. In some aspects, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The terms “transcription start site” and transcription initiation site” may be used interchangeably to refer herein to the 5′ end of a gene sequence (e.g., DNA sequence) where RNA polymerase (e.g., DNA-directed RNA polymerase) begins synthesizing the RNA transcript. The transcription start site may be the first nucleotide of a transcribed DNA sequence where RNA polymerase begins synthesizing the RNA transcript. A skilled artisan can determine a transcription start site via routine experimentation and analysis, for example, by performing a run-off transcription assay or by definitions according to FANTOMS database.

The term “promoter” as used herein refers to a region of DNA that initiates transcription of a particular gene. Promoters are typically located near the transcription start site of a gene, upstream of the gene and on the same strand (i.e., 5′ on the sense strand) on the DNA. Promoters may be about 100 to about 1000 base pairs in length.

The term “enhancer” as used herein refers to a region of DNA that may be bound by proteins (e.g., transcription factors) to increase the likelihood that transcription of a gene will occur. Enhancers may be about 50 to about 1500 base pairs in length. Enhancers may be located downstream or upstream of the transcription initiation site that it regulates and may be several hundreds of base pairs away from the transcription initiation site.

The term “silencer” as used herein refers to a DNA sequence capable of binding transcription regulation factors known as repressors, thereby negatively effecting transcription of a gene. Silencer DNA sequences may be found at many different positions throughout the DNA, including, but not limited to, upstream of a target gene for which it acts to repress transcription of the gene (e.g., silence gene expression).

A “guide RNA” or “gRNA” as provided herein refers to any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CHL respectively). The paratope or antigen-binding site is formed on the N-terminus of the antigen binding domain. The two variable domains of an antigen binding domain typically bind the epitope on an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to VH-CH1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980 , WO 91/00360; WO 92/200373; and EP 03089).

The term “aptamer” as used herein refers to an oligonucleotide or peptide molecule that binds to a specific target molecule. The target molecule may be expressed on the surface of a cell or inside a cell. In embodiments, the target molecule may form part of nucleic acid or a protein.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length. The present invention includes polypeptides that are substantially identical to any of SEQ ID NOs:1, 2, 3, 4, and 5.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The words “complementary” or “complementarity” refer to the ability of a nucleic acid in a polynucleotide to form a base pair with another nucleic acid in a second polynucleotide. For example, the sequence A-G-T is complementary to the sequence T-C-A. Complementarity may be partial, in which only some of the nucleic acids match according to base pairing, or complete, where all the nucleic acids match according to base pairing.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

“Contacting” is used in accordance with its plain ordinary meaning and refers to the process of allowing at least two distinct species (e.g. nucleic acids and/or proteins) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two or more species to react, interact, or physically touch (e.g., bind), wherein the two or more species may be, for example, a biological sample described herein and an oncogene binding agent as described herein. In embodiments, contacting includes, for example, allowing an oncogene binding agent and an amplified extrachromosomal oncogene to contact one another to form an amplified extrachromosomal oncogene binding agent complex.

As used herein, the terms “binding,” “specific binding” or “specifically binds” refer to two or more molecules forming a complex (e.g., an amplified extrachromosomal oncogene binding agent complex) that is relatively stable under physiologic conditions.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, tumor cells, metastatic cells etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” or “standard control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease (e.g., cancer) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. sarcoma, glioblastoma, lung cancer, esophageal cancer, breast cancer, bladder cancer or stomach cancer).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas that may be treated with a compound, pharmaceutical composition, or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound, pharmaceutical composition, or method provided herein include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound, pharmaceutical composition, or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, ductal carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lobular carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tubular carcinoma, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. sarcoma, glioblastoma, lung cancer, esophageal cancer, breast cancer, bladder cancer or stomach cancer)) means that the disease (e.g., cancer (e.g. sarcoma, glioblastoma, lung cancer, esophageal cancer, breast cancer, bladder cancer or stomach cancer)) is caused by (in whole or in part), or a symptom of the disease is caused by (in whole or in part) the substance or substance activity or function.

The term “prevent” refers to a decrease in the occurrence of disease symptoms in a patient. As indicated above, the prevention may be complete (no detectable symptoms) or partial, such that fewer symptoms are observed than would likely occur absent treatment.

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds of the invention can be administered alone or can be coadministered to the patient.

Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

Cancer model organism, as used herein, is an organism exhibiting a phenotype indicative of cancer, or the activity of cancer causing elements, within the organism. The term cancer is defined above. A wide variety of organisms may serve as cancer model organisms, and include for example, cancer cells and mammalian organisms such as rodents (e.g. mouse or rat) and primates (such as humans). Cancer cell lines are widely understood by those skilled in the art as cells exhibiting phenotypes or genotypes similar to in vivo cancers. Cancer cell lines as used herein includes cell lines from animals (e.g. mice) and from humans.

An “anticancer agent” as used herein refers to a molecule (e.g. compound, peptide, protein, nucleic acid, antibody) used to treat cancer through destruction or inhibition of cancer cells or tissues. Anticancer agents may be selective for certain cancers or certain tissues. In embodiments, anticancer agents herein are poly ADP ribose polymerase (PARP) inhibitors.

“Selective” or “selectivity” or the like of a compound refers to the compound's ability to discriminate between molecular targets (e.g. a compound having selectivity toward PARP).

“Specific”, “specifically”, “specificity”, or the like of a compound refers to the compound's ability to cause a particular action, such as inhibition, to a particular molecular target with minimal or no action to other proteins in the cell (e.g. a compound having specificity towards a specific PARP (e.g., PARP1, PARP2, PARP3 etc.) displays inhibition of the activity of that specific PARP ((e.g., PARP1, PARP2, PARP3 etc.), whereas the same compound displays little-to-no inhibition of other PARPs (e.g., PARP2, PARP3, PARP4 etc.).

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

A “detectable agent” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ab, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

Radioactive substances (e.g., radioisotopes) that may be used as imaging and/or labeling agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ab, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, and ²²⁵Ac. Paramagnetic ions that may be used as additional imaging agents in accordance with the embodiments of the disclosure include, but are not limited to, ions of transition and lanthanide metals (e.g. metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

A “labeled protein or polypeptide”, “labeled nucleic acid”, or “labeled peptide nucleic acid” is one that is bound, either covalently, through a linker or a chemical bond, or non-covalently, through ionic, van der Waals, electrostatic, or hydrogen bonds to a label such that the presence of the labeled protein or polypeptide, nucleic acid or peptide nucleic acid, may be detected by detecting the presence of the label bound to the labeled protein or polypeptide, nucleic acid or peptide nucleic acid. Alternatively, methods using high affinity interactions may achieve the same results where one of a pair of binding partners binds to the other, e.g., biotin, streptavidin.

The term “EGFR” or “EGFR protein” as provided herein includes any of the recombinant or naturally-occurring forms of the epidermal growth factor receptor (EGFR) or variants or homologs thereof that maintain EGFR activity (e.g. within at least 50%, 80%, 90%, 95%, 96%, 97%, 98%, 99% or 100% activity compared to EGFR). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring EGFR. In embodiments, EGFR is the protein as identified by the NCBI sequence reference GI: 29725609, homolog or functional fragment thereof.

The term “c-Myc” as provided herein includes any of the recombinant or naturally-occurring forms of the cancer Myelocytomatosis (c-Myc) or variants or homologs thereof that maintain c-Myc activity (e.g. within at least 50%, 80%, 90%, 95%, 96%, 97%, 98%, 99% or 100% activity compared to c-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring c-Myc. In embodiments, c-Myc is the protein as identified by Accession No. Q6LBK7, homolog or functional fragment thereof.

The terms “N-Myc” as provided herein includes any of the recombinant or naturally-occurring forms of the N-myc proto-oncogene protein (N-Myc) or variants or homologs thereof that maintain N-Myc activity (e.g. within at least 50%, 80%, 90%, 95%, 96%, 97%, 98%, 99% or 100% activity compared to N-Myc). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring N-Myc. In embodiments, N-Myc is the protein as identified by Accession No. P04198, homolog or functional fragment thereof.

The terms “cyclin D1” as provided herein includes any of the recombinant or naturally-occurring forms of the cyclin D1 protein (cyclin D1) or variants or homologs thereof that maintain cyclin D1 activity (e.g. within at least 50%, 80%, 90%, 95%, 96%, 97%, 98%, 99% or 100% activity compared to cyclin D1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring cyclin D1. In embodiments, cyclin D1 is the protein as identified by Accession No. P24385, homolog or functional fragment thereof.

The terms “ErbB2”, or “erythroblastic oncogene B,” as provided herein includes any of the recombinant or naturally-occurring forms of the receptor tyrosine-protein kinase erbB-2 (ErbB2) or variants or homologs thereof that maintain ErbB2 activity (e.g. within at least 50%, 80%, 90%, 95%, 96%, 97%, 98%, 99% or 100% activity compared to ErbB2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring ErbB2. In embodiments, ErbB2 is the protein as identified by Accession No. P04626, homolog or functional fragment thereof.

The terms “CDK4”, or “cyclin-dependent kinase 4” as provided herein includes any of the recombinant or naturally-occurring forms of the cyclin dependent kinase 4 (CDK4) or variants or homologs thereof that maintain CDK4 activity (e.g. within at least 50%, 80%, 90%, 95%, 96%, 97%, 98%, 99% or 100% activity compared to CDK4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK4. In embodiments, CDK4is the protein as identified by Accession No. P11802, homolog or functional fragment thereof.

The terms “CDK6”, or “cyclin-dependent kinase 6” as provided herein includes any of the recombinant or naturally-occurring forms of the cyclin dependent kinase 6 (CDK6) or variants or homologs thereof that maintain CDK6 activity (e.g. within at least 50%, 80%, 90%, 95%, 96%, 97%, 98%, 99% or 100% activity compared to CDK6). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CDK6. In embodiments, CDK6 is the protein as identified by Accession No. Q00534, homolog or functional fragment thereof.

The terms “BRAF” as provided herein includes any of the recombinant or naturally-occurring forms of the serine/threonine-protein kinase B-Raf (BRAF) or variants or homologs thereof that maintain BRAF activity (e.g. within at least 50%, 80%, 90%, 95%, 96%, 97%, 98%, 99% or 100% activity compared to BRAF). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring BRAF. In embodiments, BRAF is the protein as identified by Accession No. P15056, homolog or functional fragment thereof.

The terms “MDM2”, or “mouse double minute 2” as provided herein includes any of the recombinant or naturally-occurring forms of the mouse double minute 2 homolog (MDM2) or variants or homologs thereof that maintain MDM2 activity (e.g. within at least 50%, 80%, 90%, 95%, 96%, 97%, 98%, 99% or 100% activity compared to MDM2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM2. In embodiments, MDM2 is the protein as identified by Accession No. Q00987, homolog or functional fragment thereof.

The terms “MDM4”, or “mouse double minute 4” as provided herein includes any of the recombinant or naturally-occurring forms of the mouse double minute 4 homolog (MDM4) or variants or homologs thereof that maintain MDM4 activity (e.g. within at least 50%, 80%, 90%, 95%, 96%, 97%, 98%, 99% or 100% activity compared to MDM4). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring MDM4. In embodiments, MDM4 is the protein as identified by Accession No. 015151, homolog or functional fragment thereof.

The terms “FGFR2” as provided herein, also known as CD332 (cluster of differentiation 332), includes any of the recombinant or naturally-occurring forms of the fibroblast growth factor receptor 2 (FGFR2) or variants or homologs thereof that maintain FGFR2 activity (e.g. within at least 50%, 80%, 90%, 95%, 96%, 97%, 98%, 99% or 100% activity compared to FGFR2). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring FGFR2. In embodiments, FGFR2 is the protein as identified by UniProt accession number P21802, homolog or functional fragment thereof.

The terms “PDGFRA” as provided herein includes any of the recombinant or naturally-occurring forms of the Platelet-derived growth factor receptor alpha (PDGFRA) or variants or homologs thereof that maintain PDGFRA activity (e.g. within at least 50%, 80%, 90%, 95%, 96%, 97%, 98%, 99% or 100% activity compared to PDGFRA). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PDGFRA. In embodiments, PDGFRA is the protein as identified by UniProt accession number P16234, homolog or functional fragment thereof.

The terms “c-Met” or “c-Met protein” as provided herein, also known as tyrosine-protein kinase Met or hepatocyte growth factor receptor (HGFR), includes any of the recombinant or naturally-occurring forms of c-Met protein or variants or homologs thereof that maintain c-Met protein activity (e.g. within at least 50%, 80%, 90%, 95%, 96%, 97%, 98%, 99% or 100% activity compared to c-Met protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring c-Met protein. In embodiments, c-Met is the protein as identified by UniProt accession number P08581, homolog or functional fragment thereof.

The terms “KRAS” or “KRAS protein” as provided herein, includes any of the recombinant or naturally-occurring forms of KRAS GTPAse protein or variants or homologs thereof that maintain KRAS protein activity (e.g. within at least 50%, 80%, 90%, 95%, 96%, 97%, 98%, 99% or 100% activity compared to KRAS protein). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring KRAS protein. In embodiments, KRAS is the protein as identified by UniProt accession number P01116, homolog or functional fragment thereof.

II. Methods of Treatment

Provided herein are, inter alia, methods of treating cancer in a subject having or being at risk of developing cancer, wherein the subject has an amplified extrachromosomal oncogene. The amplified extrachromosomal oncogene present in the subject (e.g., in a cancer cell) may form part of a circular extrachromosomal DNA. The treatment methods provided herein target cancer cells that include extrachromosomal DNA by administering a therapeutically effective amount of a DNA repair pathway inhibitor thereby destabilizing the extrachromosomal DNA and promoting apoptosis of the cancer cell including the same. The unique molecular composition and physical structure of the extrachromosomal DNA in a subject's cancer cells allows for personalized cancer treatment.

In one aspect, a method of treating cancer in a human subject having or being at risk of developing cancer is provided. The method includes administering to the human subject an effective amount of a DNA repair pathway inhibitor, thereby treating cancer in the subject, wherein the human subject has an amplified extrachromosomal oncogene. In another aspect, a method of treating cancer in a human subject having or being at risk of developing cancer is provided. The method includes administering to the human subject an effective amount of a DNA repair pathway inhibitor, thereby treating cancer in the subject, wherein the human subject has been identified as having an amplified extrachromosomal oncogene.

A “DNA repair pathway inhibitor” as provided herein refers to a substance capable of detectably lowering expression of or activity level of components (e.g., protein or nucleic acids) of the DNA repair pathway compared to a control. The inhibited expression or activity of components of the DNA repair pathway can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control.

An “inhibitor” is a compound or small molecule that inhibits the DNA repair pathway e.g., by binding, partially or totally blocking stimulation of the DNA repair pathway, decrease, prevent, or delay activation of the DNA repair pathway, or inactivate, desensitize, or down-regulate signal transduction, gene expression or enzymatic activity of the DNA repair pathway. In embodiments, the DNA repair pathway inhibitor inhibits DNA repair activity or expression of DNA repair proteins. In embodiments, the DNA repair pathway inhibitor is a compound or a small molecule. In embodiments, the DNA repair pathway inhibitor is an antibody. In embodiments, the DNA repair pathway inhibitor is an antisense nucleic acid.

According to the methods provided herein, the subject is administered an effective amount of one or more of the agents (e.g., a DNA repair pathway inhibitor) provided herein. An “effective amount” is an amount sufficient to accomplish a stated purpose (e.g. achieve the effect for which it is administered, treat a disease (e.g., cancer), reduce receptor signaling activity, reduce one or more symptoms of a disease or condition). An example of an “effective amount” is an amount sufficient to contribute to the treatment, prevention, or reduction of a symptom or symptoms of a disease (e.g., cancer), which could also be referred to as a “therapeutically effective amount.” A “reduction” of a symptom or symptoms (and grammatical equivalents of this phrase) means decreasing of the severity or frequency of the symptom(s), or elimination of the symptom(s). Guidance can be found in the literature for appropriate dosages for given classes of pharmaceutical products. For example, for the given parameter, a therapeutically effective amount will show an increase or decrease of at least 5%, 10%, 15%, 20%, 25%, 40%, 50%, 60%, 75%, 80%, 90%, or at least 100%. In embodiments, this increase or decrease for a given parameter may vary throughout the day (e.g. a peak percentage increase or decrease may differ from a percentage increase or decrease when therapeutic concentrations in circulating blood are at their peak or trough concentrations dependent on daily dosing patterns and individual pharmacokinetics). Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As defined herein, the term “inhibition”, “inhibit”, “inhibiting” and the like in reference to a protein-inhibitor interaction means negatively affecting (e.g. decreasing) the activity or function of the protein or nucleic acid (e.g., amplified extrachromosomal oncogene or circular extrachromosomal DNA) relative to the activity or function of the protein or nucleic acid (e.g., amplified extrachromosomal oncogene or circular extrachromosomal DNA) in the absence of the inhibitor. In embodiments inhibition means negatively affecting (e.g. decreasing) the concentration or levels of a protein or nucleic acid (e.g., amplified extrachromosomal oncogene or circular extrachromosomal DNA) relative to the concentration or level of the protein or nucleic acid in the absence of the inhibitor. In embodiments, inhibition refers to reduction of a disease or symptoms of disease. In embodiments, inhibition refers to a reduction in the activity of a particular protein target or the level of a target nucleic acid (e.g., amplified extrachromosomal oncogene or circular extrachromosomal DNA). Thus, inhibition includes, at least in part, partially or totally blocking stimulation, decreasing, preventing, or delaying activation, or inactivating, desensitizing, or down-regulating signal transduction or enzymatic activity or the amount of a protein or nucleic acid (e.g., amplified extrachromosomal oncogene or circular extrachromosomal DNA). In embodiments, inhibition refers to a reduction of activity of a target protein resulting from a direct interaction (e.g. an inhibitor binds to the target protein). In embodiments, inhibition refers to a reduction of activity of a target protein or nucleic acid (e.g., amplified extrachromosomal oncogene or circular extrachromosomal DNA) from an indirect interaction (e.g. inhibitor binds to a protein that is involved in extrachromosomal oncogene amplification or circular extrachromosomal DNA replication, thereby preventing extrachromosomal oncogene amplification or circular extrachromosomal DNA replication).

An “ecDNA inhibitor” or “extrachromosomal DNA inhibitor” is an agent (e.g., a compound, small molecule, nucleic acid, protein) that negatively affects (e.g. decreases) the activity or function of ecDNA relative to the activity or function of ecDNA in the absence of the inhibitor. An ecDNA inhibitor as provided herein is a compound capable of reducing (decreasing) extrachromosomal oncogene amplification or circular extrachromosomal DNA replication relative to the absence of the inhibitor. In embodiments, the ecDNA inhibitor is a DNA repair pathway inhibitor.

The term “extrachromosomal DNA” or “ecDNA” as used herein, refers to a deoxyribonucleotide polymer of chromosomal composition (i.e. includes histone proteins) that does not form part of a cellular chromosome. ecDNA molecules have a circular structure and are not linear, as compared to cellular chromosomes. ecDNA may be found outside of the nucleus of a cell and may therefore also referred to as extranuclear DNA or cytoplasmic DNA. Circular extrachromosomal DNA (ecDNA) may be derived from genomic DNA, and may include repetitive sequences of DNA found in both coding and non-coding regions of chromosomes. The formation of ecDNA may occur independently of the cellular replication process. EcDNA may have a size from about 500,000 base pairs to about 5,000,000 base pairs.

In embodiments, the circular extrachromosomal DNA includes about 250,000 base pairs to about 10,000,000 base pairs. In embodiments, the circular extrachromosomal DNA includes about 500,000 base pairs to about 10,000,000 base pairs. In embodiments, the circular extrachromosomal DNA includes about 750,000 base pairs to about 10,000,000 base pairs. In embodiments, the circular extrachromosomal DNA includes about 1,000,000 base pairs to about 10,000,000 base pairs. In embodiments, the circular extrachromosomal DNA includes about 1,250,000 base pairs to about 10,000,000 base pairs. In embodiments, the circular extrachromosomal DNA includes about 1,500,000 base pairs to about 10,000,000 base pairs. In embodiments, the circular extrachromosomal DNA includes about 1,750,000 base pairs to about 10,000,000 base pairs. In embodiments, the circular extrachromosomal DNA includes about 2,000,000 base pairs to about 10,000,000 base pairs. In embodiments, the circular extrachromosomal DNA includes about 2,250,000 base pairs to about 10,000,000 base pairs. In embodiments, the circular extrachromosomal DNA includes about 2,500,000 base pairs to about 10,000,000 base pairs. In embodiments, the circular extrachromosomal DNA includes about 2,750,000 base pairs to about 10,000,000 base pairs. In embodiments, the circular extrachromosomal DNA includes about 3,000,000 base pairs to about 10,000,000 base pairs. In embodiments, the circular extrachromosomal DNA includes about 3,250,000 base pairs to about 10,000,000 base pairs. In embodiments, the circular extrachromosomal DNA includes about 3,500,000 base pairs to about 10,000,000 base pairs. In embodiments, the circular extrachromosomal DNA includes about 3,750,000 base pairs to about 10,000,000 base pairs. In embodiments, the circular extrachromosomal DNA includes about 4,000,000 base pairs to about 10,000,000 base pairs. In embodiments, the circular extrachromosomal DNA includes about 4,250,000 base pairs to about 10,000,000 base pairs. In embodiments, the circular extrachromosomal DNA includes about 4,500,000 base pairs to about 10,000,000 base pairs. In embodiments, the circular extrachromosomal DNA includes about 4,750,000 base pairs to about 10,000,000 base pairs.

In embodiments, the circular extrachromosomal DNA includes 250,000 base pairs to 10,000,000 base pairs. In embodiments, the circular extrachromosomal DNA includes 500,000 base pairs to 10,000,000 base pairs. In embodiments, the circular extrachromosomal DNA includes 750,000 base pairs to 10,000,000 base pairs. In embodiments, the circular extrachromosomal DNA includes 1,000,000 base pairs to 10,000,000 base pairs. In embodiments, the circular extrachromosomal DNA includes 1,250,000 base pairs to 10,000,000 base pairs. In embodiments, the circular extrachromosomal DNA includes 1,500,000 base pairs to 10,000,000 base pairs. In embodiments, the circular extrachromosomal DNA includes 1,750,000 base pairs to 10,000,000 base pairs. In embodiments, the circular extrachromosomal DNA includes 2,000,000 base pairs to 10,000,000 base pairs. In embodiments, the circular extrachromosomal DNA includes 2,250,000 base pairs to 10,000,000 base pairs. In embodiments, the circular extrachromosomal DNA includes 2,500,000 base pairs to 10,000,000 base pairs. In embodiments, the circular extrachromosomal DNA includes 2,750,000 base pairs to 10,000,000 base pairs. In embodiments, the circular extrachromosomal DNA includes 3,000,000 base pairs to 10,000,000 base pairs. In embodiments, the circular extrachromosomal DNA includes 3,250,000 base pairs to 10,000,000 base pairs. In embodiments, the circular extrachromosomal DNA includes 3,500,000 base pairs to 10,000,000 base pairs. In embodiments, the circular extrachromosomal DNA includes 3,750,000 base pairs to 10,000,000 base pairs. In embodiments, the circular extrachromosomal DNA includes 4,000,000 base pairs to 10,000,000 base pairs. In embodiments, the circular extrachromosomal DNA includes 4,250,000 base pairs to 10,000,000 base pairs. In embodiments, the circular extrachromosomal DNA includes 4,500,000 base pairs to 10,000,000 base pairs. In embodiments, the circular extrachromosomal DNA includes 4,750,000 base pairs to 10,000,000 base pairs. In embodiments, the circular extrachromosomal DNA includes 500,000 base pairs. In embodiments, the circular extrachromosomal DNA includes 1,300,000 base pairs. In embodiments, the circular extrachromosomal DNA includes 250000, 500000, 750000, 1000000, 1250000, 1500000, 1750000, 2000000, 2250000, 2500000, 2750000, 3000000, 3250000, 3500000, 3750000, 4000000, 4250000, 4500000, 4750000, or 10000000 base pairs. Where the circular extrachromosomal DNA includes 250000, 500000, 750000, 1000000, 1250000, 1500000, 1750000, 2000000, 2250000, 2500000, 2750000, 3000000, 3250000, 3500000, 3750000, 4000000, 4250000, 4500000, 4750000, or 10000000 base pairs, the circular extrachromosomal DNA is 250000, 500000, 750000, 1000000, 1250000, 1500000, 1750000, 2000000, 2250000, 2500000, 2750000, 3000000, 3250000, 3500000, 3750000, 4000000, 4250000, 4500000, 4750000, or 10000000 nucleotides in length.

As used herein, the term “oncogene” is a term well known in the art and used according to its conventional meaning in the art. An oncogene is a gene capable of predisposing a cell to cancer due to the presence of one or more mutations in said gene or due to increased expression levels of said gene relative to its expression levels in a healthy cell. The terms “amplified oncogene” or “oncogene amplification” refer to an oncogene or fragment thereof being present in multiple copy numbers (e.g., at least 2 or more) in a chromosome. Likewise, an “amplified extrachromosomal oncogene” is an oncogene or fragment thereof, which is present in multiple copy numbers and the multiple copies of said oncogene or fragment thereof form part of an extrachromosomal DNA molecule. In embodiments, the oncogene forms part of an extrachromosomal DNA. In embodiments, the amplified oncogene forms part of an extrachromosomal DNA. In embodiments, the amplified extrachromosomal oncogene is EGFR, c-Myc, N-Myc, cyclin D1, ErbB2, CDK4, CDK6, BRAF, MDM2, or MDM4. In embodiments, the extrachromosomal oncogene is EGFR. In embodiments, the extrachromosomal oncogene is c-Myc. In embodiments, the extrachromosomal oncogene is N-Myc. In embodiments, the extrachromosomal oncogene is cyclin D1. In embodiments, the extrachromosomal oncogene is ErbB2. In embodiments, the extrachromosomal oncogene is CDK4. In embodiments, the extrachromosomal oncogene is CDK6. In embodiments, the extrachromosomal oncogene is BRAF. In embodiments, the extrachromosomal oncogene is MDM2. In embodiments, the extrachromosomal oncogene is MDM4. In embodiments, the amplified extrachromosomal oncogene is FGFR2, PDGFRA, c-MET, or KRAS. In embodiments, the amplified extrachromosomal oncogene is FGFR2. In embodiments, the amplified extrachromosomal oncogene is PDGFRA. In embodiments, the amplified extrachromosomal oncogene is c-MET. In embodiments, the amplified extrachromosomal oncogene is KRAS.

According to the methods provided herein including embodiments thereof, a human subject that has been identified as having an amplified extrachromosomal oncogene is identified prior to the administering, by detecting an amplified extrachromosomal oncogene in a cancer cell in a first biological sample obtained from the human subject by contacting the biological sample with an oncogene-binding agent and detecting binding of the oncogene-binding agent to the amplified extrachromosomal oncogene. Any of the methods described in Turner (Nature, 2017 Mar. 2; 543(7643): 122-125. doi: 10.1038/nature21356), which is incorporated herewith in its entirety and for all purposes, may be used for the detection steps provided herein including embodiments thereof (e.g., of an amplified extrachromosomal oncogene or the circular extrachromosomal DNA). In embodiments, the method may include a step of detecting an amplified extrachromosomal oncogene, a level of a circular extrachromosomal DNA or a level of heterogeneity thereof in a cancer cell in a first biological sample obtained from the human subject prior to the administering of the DNA repair pathway inhibitor.

The methods provided herein including embodiments thereof, may include a step of detecting an amplified extrachromosomal oncogene, a level of a circular extrachromosomal DNA or a level of heterogeneity thereof in a cancer cell prior to the administering of the DNA repair pathway inhibitor. Thus, in embodiments, the method includes prior to the administering, detecting an amplified extrachromosomal oncogene in a cancer cell in a first biological sample obtained from the human subject by contacting the biological sample with an oncogene-binding agent and detecting binding of the oncogene-binding agent to the amplified extrachromosomal oncogene. Any of the methods described in Turner (Nature, 2017 Mar. 2; 543(7643): 122-125. doi: 10.1038/nature21356), which is incorporated herewith in its entirety and for all purposes, may be used for the detection steps provided herein including embodiments thereof (e.g., of an amplified extrachromosomal oncogene or the circular extrachromosomal DNA).

In one aspect, a method of treating cancer in a human subject in need thereof is provided. The method includes (i) detecting an amplified extrachromosomal oncogene in a cancer cell in a first biological sample obtained from a human subject having or being at risk of developing cancer by contacting the biological sample with an oncogene-binding agent and detecting binding of the oncogene-binding agent to the amplified extrachromosomal oncogene; and (ii) administering to the human subject an effective amount of a DNA repair pathway inhibitor thereby treating cancer in the subject.

An “oncogene-binding agent” as provided herein refers to a substance capable of binding an amplified extrachromosomal oncogene. The oncogene-binding agent may bind the amplified extrachromosomal oncogene either covalently, through a linker or a chemical bond, or non-covalently, through ionic, van der Waals, electrostatic, or hydrogen bonds. Upon binding of the oncogene-binding agent to the amplified extrachromosomal oncogene an amplified extrachromosomal oncogene binding agent complex is formed. The methods provided herein including embodiments thereof include detecting the amplified extrachromosomal oncogene binding agent complex, thereby detecting the amplified extrachromosomal oncogene in a biological sample.

The oncogene-binding agent may bind the amplified extrachromosomal oncogene either covalently, through a linker or a chemical bond, or non-covalently, through ionic, van der Waals, electrostatic, or hydrogen bonds to a label such that the presence of the amplified extrachromosomal oncogene may be detected by detecting the presence of the label bound to the oncogene-binding agent.

The oncogene-binding agent may be a nucleic acid (e.g., DNA or RNA) capable of hybridizing to the amplified extrachromosomal oncogene or a portion thereof. The oncogene-binding agent may be a protein capable of binding to the amplified extrachromosomal oncogene or a portion thereof. Alternatively, the oncogene-binding agent may be a protein capable of binding to a protein (e.g., a histone protein) bound to the amplified extrachromosomal oncogene or a portion thereof. In embodiments, the oncogene-binding agent binds a nucleic acid modification (e.g., a nucleic acid methylation) or a modification of a protein (e.g., methylation, acetylation, phosphorylation) bound to the oncogene-binding agent. The oncogene-binding agent may be a nucleic acid or a protein. In embodiments, the oncogene-binding agent is a nucleic acid. In embodiments, the oncogene-binding agent is a peptide. In embodiments, the oncogene-binding agent is a peptide nucleic acid. In embodiments, the oncogene-binding agent is a small molecule. In embodiments, the oncogene-binding agent is an antibody. In embodiments, the oncogene-binding agent is a nucleic acid, a peptide nucleic acid or a protein. In embodiments, the oncogene-binding agent is a nucleic acid. In embodiments, the oncogene-binding agent is a peptide nucleic acid. In embodiments, the oncogene-binding agent is a protein. In embodiments, the oncogene-binding agent is a labeled nucleic acid, a labeled peptide nucleic acid or a labeled protein. In embodiments, the oncogene-binding agent is a labeled nucleic acid. In embodiments, the oncogene-binding agent is a labeled peptide nucleic acid. In embodiments, the oncogene-binding agent is a labeled protein.

In embodiments, the amplified extrachromosomal oncogene is contacted with an oncogene-binding agent in a biological sample (e.g., whole blood, serum or plasma). In embodiments, the oncogene-binding agent includes a detectable moiety. In embodiments, the detectable moiety is a fluorescent moiety. In embodiments, the oncogene-binding agent includes a capturing moiety. A “capturing moiety” refers to a protein or nucleic acid, which is covalently, through a linker or a chemical bond, or non-covalently attached to the oncogene-binding agent and is capable of interacting with a capturing agent. In embodiments, the oncogene-binding agent includes a detectable moiety. In embodiments, the detectable moiety is a fluorescent moiety. In embodiments, the oncogene-binding agent includes a capturing moiety. A “capturing moiety” refers to a protein or nucleic acid, which is covalently, through a linker or a chemical bond, or non-covalently attached to the oncogene-binding agent and is capable of interacting with a capturing agent. An example of a capturing moiety useful for the methods provided herein is biotin. In embodiments, the capturing moiety is biotin. In embodiments, the capturing moiety is a cleavable capturing moiety. In embodiments, the capturing moiety is photocleavable biotin.

A “capturing agent” as provided herein refers to an agent capable of binding a capturing moiety. The interaction between the capturing moiety and the capturing agent may be a high affinity interaction, wherein the capturing moiety and the capturing agent bind to each other (e.g., biotin, streptavidin). An example of a capturing agent useful for the methods provided herein are streptavidin coated beads. In embodiments, the capturing agent is a streptavidin coated bead. Without limitation any suitable affinity binding pairs known in the art may be used as capturing moiety and capturing agent in the methods provided herein. For example, the capturing moiety may be an antibody and the capturing agent may be an antigen-coated bead. In embodiments, the capturing moiety is biotin and the capturing agent is a streptavidin coated bead.

The amplified extrachromosomal oncogene binding agent complex may be separated from the sample and unbound components contained therein by contacting the amplified extrachromosomal oncogene binding agent complex with a capturing agent as described above (e.g., streptavidin-coated beads). Thus, in embodiments, the detecting includes contacting the amplified extrachromosomal oncogene binding agent complex with a capturing agent, thereby forming a captured amplified extrachromosomal oncogene binding agent complex. The captured amplified extrachromosomal oncogene binding agent complex may be washed to remove any unbound components.

The detected amplified extrachromosomal oncogene may form part of a circular extrachromosomal DNA and the detecting performed in the methods provided herein may include detecting a level of the circular extrachromosomal DNA relative to a standard control. In embodiments, the detecting includes detecting a level of the circular extrachromosomal DNA relative to a standard control. In embodiments, the detecting includes detecting a level of the amplified extrachromosomal oncogene relative to a standard control. A level of the amplified extrachromosomal oncogene may be the amount of oncogene copies or fragments thereof present on a circular extrachromosomal DNA relative to a standard control. In embodiments, the level of the amplified extrachromosomal oncogene is increased relative to a standard control In embodiments, the amount of oncogene copies or fragments thereof present on a circular extrachromosomal DNA is increased relative to a standard control.

“A level of the circular extrachromosomal DNA” as referred to herein is the amount of circular extrachromosomal DNA molecules detectable in a cell. A circular extrachromosomal DNA as provided herein may be a single molecule of an extrachromosomal DNA consisting of a double-stranded DNA associated to histone proteins or it may be a complex formed by individual molecules. Thus, a level of circular extrachromosomal DNA includes the amount of individual circular extrachromosomal DNA molecules as well as complexes thereof. The Circular extrachromosomal DNA complexes include a plurality of single circular extrachromosomal DNA molecules covalently and/or non-covalently bound to each other.

In embodiments, the detecting includes mapping the circular extrachromosomal DNA. Mapping of the circular extrachromosomal DNA may include determining the locus of genes (e.g., oncogenes) or fragments thereof and their distance relative to each other on the circular extrachromosomal DNA. Where the distance of genes or fragments thereof on the circular extrachromosomal DNA is determined, the physical distance may be determined and/or the distance based on the genetic linkage information of the genes may be determined. In embodiments, the detecting includes detecting genetic heterogeneity of the circular extrachromosomal DNA relative to a standard control.

A “standard control” as provided herein refers to a sample that serves as a reference, usually a known reference, for comparison to a test sample. For example, a test sample can be taken from a patient suspected of having a disease (e.g., cancer) or at risk of developing the disease and compared to samples from a patient known to have the disease, or a known normal (non-disease) individual. A control can also represent an average value gathered from a population of similar individuals, e.g., disease patients or healthy individuals with a similar medical background, same age, weight, etc. A control value can also be obtained from the same individual, e.g., from an earlier-obtained sample, prior to disease, or prior to treatment. One of skill will recognize that controls can be designed for assessment of any number of parameters.

One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

In some examples of the disclosed methods, when the amount of oncogene amplification, the level of a circular extrachromosomal DNA or the amount of genetic heterogeneity therein are assessed, the amount of oncogene amplification, the level of a circular extrachromosomal DNA or the amount of genetic heterogeneity is compared with a control level or amount (e.g., in a healthy subject or in an untreated subject). By control is meant the amount of oncogene amplification, the level of a circular extrachromosomal DNA or the amount of genetic heterogeneity therein in a sample or subject lacking the disease (cancer), a sample or subject at a selected stage of the disease or disease state, or in the absence of a particular variable such as a therapeutic agent. Alternatively, the control includes a known amount of oncogene amplification, level of a circular extrachromosomal DNA or a known amount of genetic heterogeneity thereof. Such a known amount correlates with an average level of subjects lacking the disease, at a selected stage of the disease or disease state, or in the absence of a particular variable such as a therapeutic agent. A control also includes the amount of oncogene amplification, the level of a circular extrachromosomal DNA or a known amount of genetic heterogeneity thereof from one or more selected samples or subjects as described herein. For example, a control includes an assessment of the amount of oncogene amplification, the level of a circular extrachromosomal DNA or the amount of genetic heterogeneity thereof in a sample from a subject that does not have the disease, is at a selected stage of disease or disease state, or has not received treatment for the disease. Another exemplary control level includes an amount of oncogene amplification, a level of a circular extrachromosomal DNA or an amount of genetic heterogeneity thereof in samples taken from multiple subjects that do not have the disease, are at a selected stage of the disease, or have not received treatment for the disease.

When the standard control is the amount of oncogene amplification, the level of a circular extrachromosomal DNA or the amount of genetic heterogeneity thereof in a sample or subject in the absence of a therapeutic agent, the control sample or subject is optionally the same sample or subject to be tested before or after treatment with a therapeutic agent or is a selected sample or subject in the absence of the therapeutic agent. Alternatively, a standard control is an average expression level calculated from a number of subjects without a particular disease. A control level also includes a known control level or value known in the art.

In embodiments, the first biological sample is a blood-derived sample, a urine-derived sample, a tumor sample, or a tumor fluid sample. In embodiments, the first biological sample is a blood-derived sample. In embodiments, the first biological sample is a urine-derived sample. In embodiments, the first biological sample is a tumor sample. In embodiments, the first biological sample is a tumor-derived sample. In embodiments, the first biological sample is a tumor fluid sample.

In embodiments, the DNA repair pathway inhibitor is a peptide, small molecule, nucleic acid, antibody or aptamer. In embodiments, the DNA repair pathway inhibitor is a peptide. In embodiments, the DNA repair pathway inhibitor is a small molecule. In embodiments, the DNA repair pathway inhibitor is a nucleic acid. In embodiments, the DNA repair pathway inhibitor is an antibody. In embodiments, the DNA repair pathway inhibitor is an aptamer. In embodiments, the DNA repair pathway inhibitor does not modulate EGFR signaling. In embodiments, the DNA repair pathway inhibitor does not inhibit EGFR signaling. In embodiments, the DNA repair pathway inhibitor is not a specific EGFR inhibitor. In embodiments, the DNA repair pathway inhibitor is not an EGFR inhibitor. In embodiments, the DNA repair pathway inhibitor does not specifically modulate EGFR stimulation. In embodiments, the DNA repair pathway inhibitor does not modulate EGFR stimulation. In embodiments, the DNA repair pathway inhibitor does not inhibit EGFR stimulation. In embodiments, the DNA repair pathway inhibitor does not modulate EGFR activity. In embodiments, the DNA repair pathway inhibitor does not inhibit EGFR activity. In embodiments, the DNA repair pathway inhibitor is not a small molecule tyrosine kinase inhibitor. In embodiments, the DNA repair pathway inhibitor is not cetuximab, gefitininb, erlotinib, laptinib, or panitumumab.

In embodiments, the DNA repair pathway inhibitor is a poly ADP ribose polymerase (PARP) inhibitor. The term “PARP” or “PARP protein” as provided herein includes any of the recombinant or naturally-occurring forms of the poly(ADP-ribose) polymerase (PARP) or variants or homologs thereof that maintain PARP activity (e.g. within at least 50%, 80%, 90%, 95%, 96%, 97%, 98%, 99% or 100% activity compared to PARP). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring PARP. In embodiments, PARP is the protein as identified by UniProtKB No. P09874 or a variant or homolog having substantial identity thereto. In embodiments, PARP is the protein as identified by UniProtKB No. Q9UGN5 or a variant or homolog having substantial identity thereto. In embodiments, PARP is the protein as identified by UniProtKB No. Q9Y6F1 or a variant or homolog having substantial identity thereto. In embodiments, PARP is the protein as identified by UniProtKB No. Q9UKK3 or a variant or homolog having substantial identity thereto. In embodiments, PARP is the protein as identified by UniProtKB No. 095271 or a variant or homolog having substantial identity thereto. In embodiments, PARP is the protein as identified by UniProtKB No. Q9H2K2 or a variant or homolog having substantial identity thereto. In embodiments, PARP is the protein as identified by UniProtKB No. Q2NL67 or a variant or homolog having substantial identity thereto. In embodiments, PARP is the protein as identified by UniProtKB No. Q7Z3E1 or a variant or homolog having substantial identity thereto. In embodiments, PARP is the protein as identified by UniProtKB No. Q8N3A8 or a variant or homolog having substantial identity thereto. In embodiments, PARP is the protein as identified by UniProtKB No. Q8IXQ6 or a variant or homolog having substantial identity thereto. In embodiments, PARP is the protein as identified by UniProtKB No. Q53GL7 or a variant or homolog having substantial identity thereto. In embodiments, PARP is the protein as identified by UniProtKB No. Q9NR21 or a variant or homolog having substantial identity thereto. In embodiments, PARP is the protein as identified by UniProtKB No. Q9HOJ9 or a variant or homolog having substantial identity thereto. In embodiments, PARP is the protein as identified by UniProtKB No. Q460N5 or a variant or homolog having substantial identity thereto. In embodiments, PARP is the protein as identified by UniProtKB No. Q460N3 or a variant or homolog having substantial identity thereto. In embodiments, PARP is the protein as identified by UniProtKB No. Q8N5Y8 or a variant or homolog having substantial identity thereto.

A “poly ADP ribose polymerase inhibitor” or “PARP inhibitor” as provided herein refers to a substance capable of detectably lowering expression of or activity level of PARP compared to a control. The inhibited expression or activity of PARP can be 10%, 20%, 30%, 40%, 50%, 60%, 70%, 80%, 90% or less than that in a control. In certain instances, the inhibition is 1.5-fold, 2-fold, 3-fold, 4-fold, 5-fold, 10-fold, or more in comparison to a control. In embodiments, the PARP inhibitor lowers expression of or activity level of PARP1, PARP2 or both. In embodiments, the PARP inhibitor lowers expression of or activity level of PARP1, PARP2, PARP3, PARP4 or any combination thereof. In embodiments, the the PARP inhibitor lowers expression of or activity level of a specific PARP (e.g., PARP1) or of two or more homologs of PARP (e.g., PARP1, PARP2, PARP3, PARP4 etc.). An “inhibitor” is a compound or small molecule that inhibits PARP e.g., by binding, partially or totally blocking stimulation of PARP, decrease, prevent, or delay activation of PARP, or inactivate, desensitize, or down-regulate signal transduction, gene expression or enzymatic activity of PARP. In embodiments, the PARP inhibitor inhibits PARP activity or expression PARP. In embodiments, the PARP inhibitor inhibits PARP activity or expression of PARP. In embodiments, the PARP inhibitor is a compound or a small molecule. In embodiments, the PARP inhibitor is an antibody. In embodiments, the PARP inhibitor is rucaparib, olaparib, niraparib, veliparib, talazoparib, CEP 9722, E7016 (GPI-21016), BGB-290, INO-1001, MP-124, or LT-00673. In embodiments, the DNA repair pathway inhibitor is rucaparib or olaparib. In embodiments, the DNA repair pathway inhibitor is rucaparib. In embodiments, the DNA repair pathway inhibitor is olaparib. In embodiments, the DNA repair pathway inhibitor is niraparib. In embodiments, the DNA repair pathway inhibitor is veliparib. In embodiments, the DNA repair pathway inhibitor is talazoparib. In embodiments, the DNA repair pathway inhibitor is CEP 9722. In embodiments, the DNA repair pathway inhibitor is E7016 (GPI-21016). In embodiments, the DNA repair pathway inhibitor is BGB-290. In embodiments, the DNA repair pathway inhibitor is INO-1001. In embodiments, the DNA repair pathway inhibitor is MP-124. In embodiments, the DNA repair pathway inhibitor is LT-00673.

The compound “rucaparib” as provided herein refers in its customary sense to the compound identified by Cas Registry Number 283173-50-2. The compound “olaparib” as provided herein refers in its customary sense to the compound identified by Cas Registry Number 763113-22-0. The compound “niraparib” as provided herein refers in its customary sense to the compound identified by Cas Registry Number 1038915-60-4. The compound “niraparib” as provided herein refers in its customary sense to the compound identified by Cas Registry Number 1038915-60-4. The compound “veliparib” as provided herein refers in its customary sense to the compound identified by PubChem CID Number 11960529. The compound “talazoparib” as provided herein refers in its customary sense to the compound identified by ChemSpider Reference Number 28637772.

In embodiments, the cancer is sarcoma, glioblastoma, lung cancer, esophageal cancer, breast cancer, bladder cancer or stomach cancer. In embodiments, the cancer is sarcoma. In embodiments, the cancer is glioblastoma. In embodiments, the cancer is lung cancer. In embodiments, the cancer is esophageal cancer. In embodiments, the cancer is breast cancer. In embodiments, the cancer is bladder cancer. In embodiments, the cancer is stomach cancer. In embodiments, the cancer is ovarian cancer. In embodiments, the cancer is head and neck cancer. In embodiments, the cancer is melanoma. In embodiments, the cancer is uveal melanoma. In embodiments, the cancer is acral melanoma. In embodiments, the cancer is diffuse large B cell lymphoma. In embodiments, the cancer is colon cancer. In embodiments, the cancer is uterine endometrial cancer. In embodiments, the cancer is cervical cancer. In embodiments, the cancer is prostate cancer. In embodiments, the cancer is renal cancer. In embodiments, the cancer is liver cancer. In embodiments, the cancer is liver hepatocellular carcinoma. In embodiments, the cancer is glioma.

The methods provided herein including embodiments thereof may be used to monitor effectiveness of treatment. Thus, in embodiments, the detecting includes detecting a first level of the amplified extrachromosomal oncogene. In embodiments, after step (ii): (iii) obtaining a second biological sample from the subject; (iv) detecting a second level of the amplified extrachromosomal oncogene; and (v) comparing the first level to the second level. In embodiments, the first biological sample is obtained at a time t₀, from the subject and the second biological sample is obtained at a later time t₁ from the subject. In embodiments, the first level of the amplified extrachromosomal oncogene is a first amount of oncogene copies or fragments thereof and the second level of the amplified extrachromosomal oncogene is a second amount of oncogene copies or fragments thereof. Where the level of the amplified extrachromosomal oncogene in the second biological sample relative to the first biological sample is decreased, the treatment is efficacious for treating cancer in the subject. Where the level of the circular extrachromosomal DNA in the second biological sample relative to the first biological sample is decreased, the treatment is efficacious for treating cancer in the subject. In embodiments, the time t₀ is before the treatment has been administered to the subject, and the time t₁ is after the treatment has been administered to the subject. In embodiments, the time t₀ is after the treatment has been administered to the subject, and the time t₁ is later than time t₀ after the treatment has been administered to the subject. In embodiments, the treatment is administered multiple times. In embodiments, the comparing is repeated for biological samples obtained from the subject over a range of times.

In one aspect, a method of treating cancer in a human subject in need thereof is provided. The method includes (i) detecting a first level of an amplified extrachromosomal oncogene in a cancer cell in a first biological sample obtained from a human subject having or being at risk of developing cancer; (ii) administering to the human subject an effective amount of a DNA repair pathway inhibitor; (iii) detecting a second level of an amplified extrachromosomal oncogene in a cancer cell in a second biological sample obtained from the human subject; and (iv) comparing the first level to the second level, thereby treating cancer in the human subject.

Any of the embodiments described for the methods above are applicable for this method. Thus, in embodiments, the detecting in step (i) and (iii) includes contacting the first and second biological sample with an oncogene-binding agent and detecting binding of the oncogene-binding agent to the amplified extrachromosomal oncogene. In embodiments, the oncogene-binding agent is a labeled nucleic acid probe. In embodiments, the amplified extrachromosomal oncogene is EGFR, c-Myc, N-Myc, cyclin D1, ErbB2, CDK4, CDK6, BRAF, MDM2, or MDM4. In embodiments, the first or second biological sample is a blood-derived sample, a urine-derived sample, a tumor sample, or a tumor fluid sample. In embodiments, the DNA repair pathway inhibitor is a peptide, small molecule, nucleic acid, antibody or aptamer. In embodiments, the DNA repair pathway inhibitor is a poly ADP ribose polymerase (PARP) inhibitor. In embodiments, the DNA repair pathway inhibitor is administered at an effective amount of about 1 μM. In embodiments, the DNA repair pathway inhibitor is administered at an effective amount of about 5 μM. In embodiments, the DNA repair pathway inhibitor is rucaparib or olaparib. In embodiments, the cancer is sarcoma, glioblastoma, lung cancer, esophageal cancer, breast cancer, bladder cancer or stomach cancer.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

### Methods of Targeting Tumors with Copy Number Alterations Based on Unique Vulnerabilities in the Processes of DNA Replication, DNA Repair and Cellular Metabolism that are Generated by the Presence of Extrachromosomal Oncogene Amplification

We recently made the discovery that the most common genetic drivers of cancer, amplified oncogenes, which are also compelling targets for drug development, are not found on their native chromosomal locus as they are shown to be on the maps produced by The Cancer Genome Atlas (TCGA) or International Genome Consortium (ICGC), but rather, on circular extrachromosomal DNA (Turner et al., Nature, 2017), enabling malignant tumors to rapidly develop, diversify and resist treatment. We reported that: 1) all of 17 different cancer types studied displayed evidence of having oncogene amplification on extrachromosomal DNA; 2) nearly half of human cancers possess amplified oncogenes on circular extrachromosomal DNA and 3) most commonly amplified oncogenes are found on circular extrachromosomal DNA. We also previously showed that reversible loss of extrachromosomal DNA is a potent mechanism by which cancer tumors resist treatment with targeted inhibitors, suggesting the need for new therapeutic approaches for nearly half of all cancers, informed by new knowledge of the unique properties of tumors containing extrachromosomal oncogene amplification.

We have identified four therapeutically exploitable vulnerabilities of cancers containing oncogenes amplified on extrachromosomal DNA and methods to attack these vulnerabilities:

1.) Method of targeting cancers with extrachromosomal (ecDNA) oncogene amplification based on using targeted agents to lower the DNA copy number of the amplified oncogenes:

2) Method of targeting cancers with extrachromosomal (ecDNA) oncogene amplification based on enhanced dependence of ecDNA on de novo nucleotide synthesis.

3.) Method of targeting cancers with extrachromosomal (ecDNA) oncogene amplification based on differential metabolic requirements including dependence on glucose.

4.) Method of targeting cancers with extrachromosomal (ecDNA) oncogene amplification based on differential DNA replication kinetics and DNA damage and repair mechanisms.

We have identified four therapeutically exploitable vulnerabilities of cancers containing oncogenes amplified on extrachromosomal DNA and methods to attack these vulnerabilities:

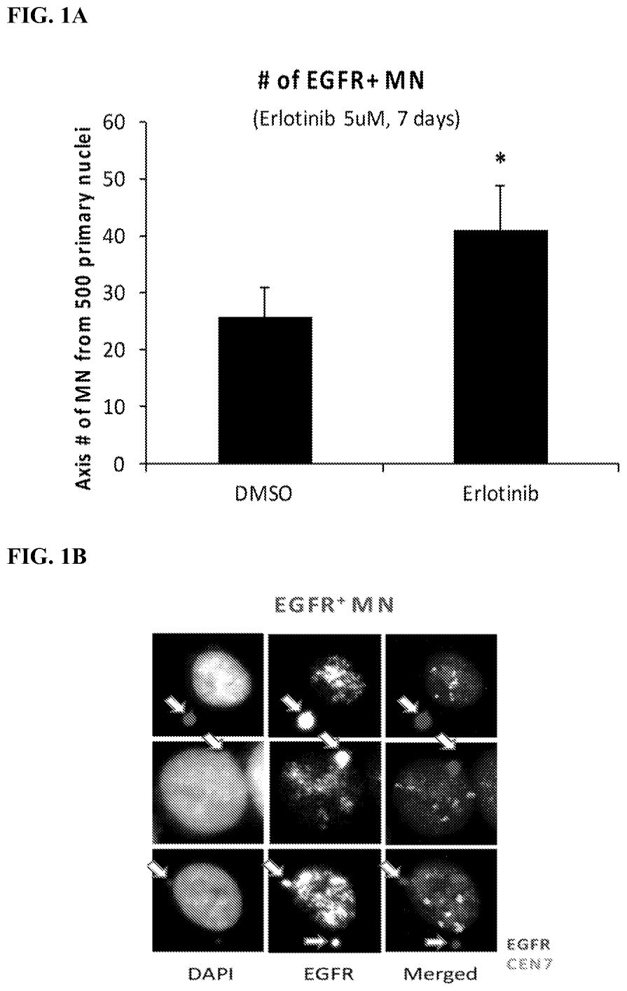

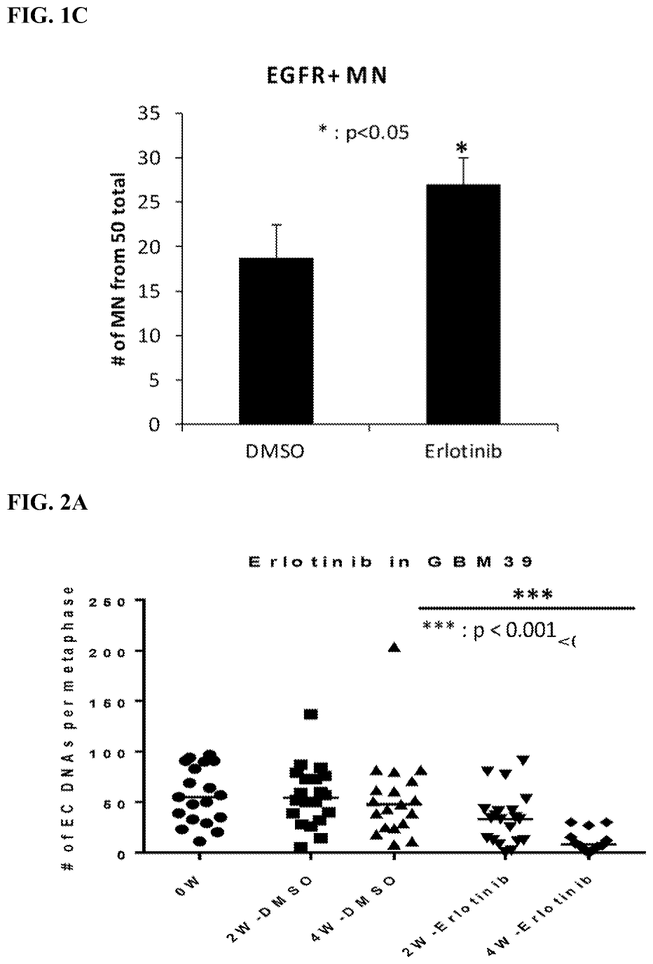

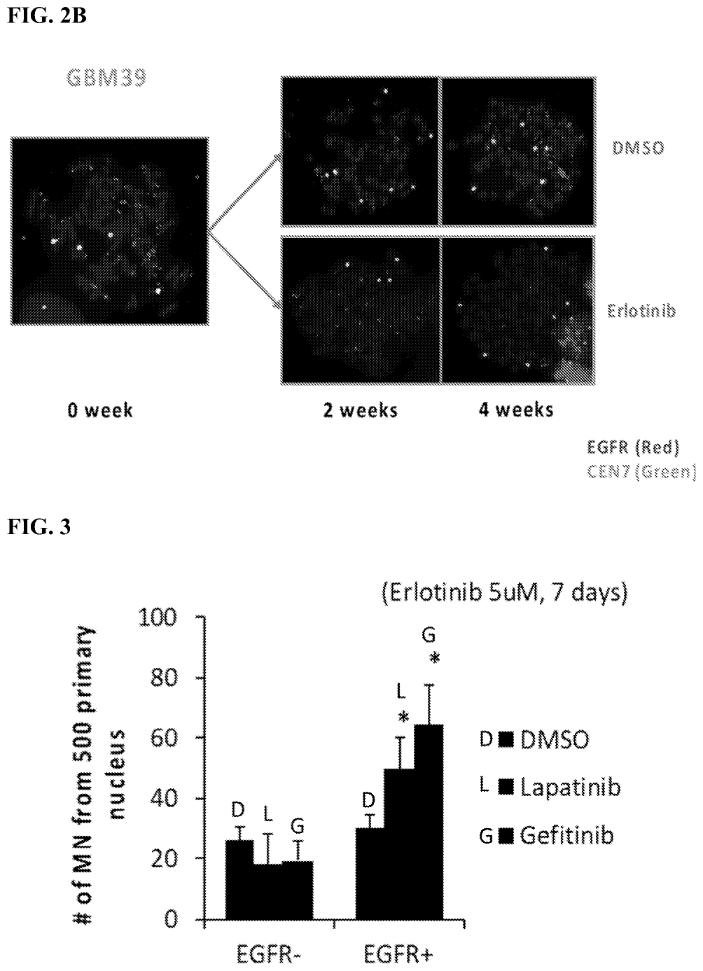

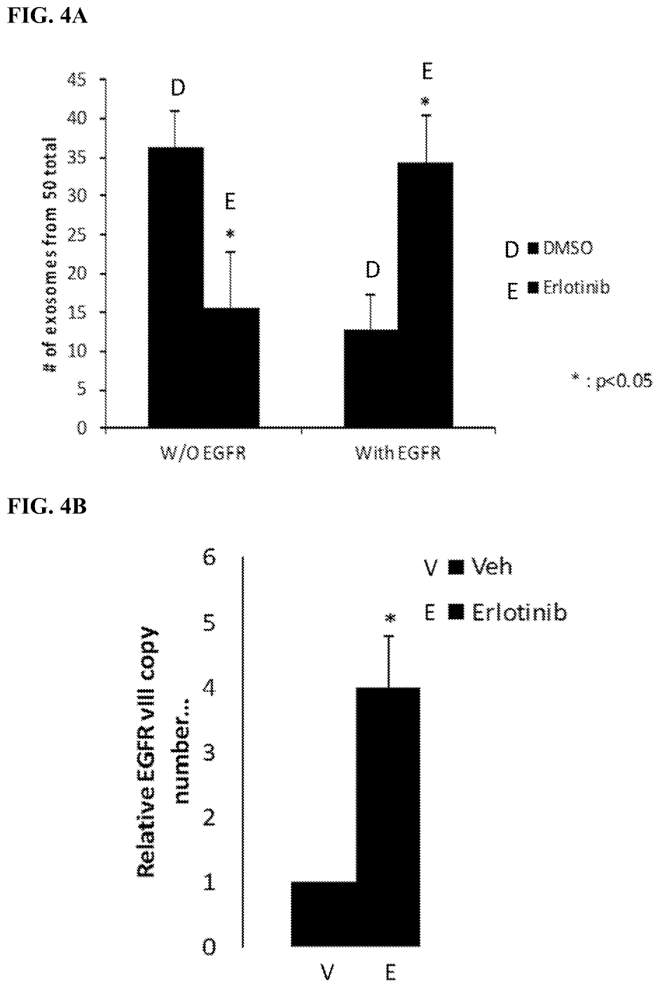

Method of targeting cancers with extrachromosomal (ecDNA) oncogene amplification based on using targeted agents to lower the DNA copy number of the amplified oncogenes: We demonstrate in patient derived glioblastoma neurosphere culture that a panel of EGFR tyrosine kinase inhibitors dramatically lowers the amount of EGFR oncogene DNA in tumor cells by causing the formation of EGFR/EGFRvIII containing micronuclei from those ecDNAs, which are then extruded from the cell in exosomes (FIGS. 1-4). We have extended these findings to a panel of other patient-derived cancer cell lines.

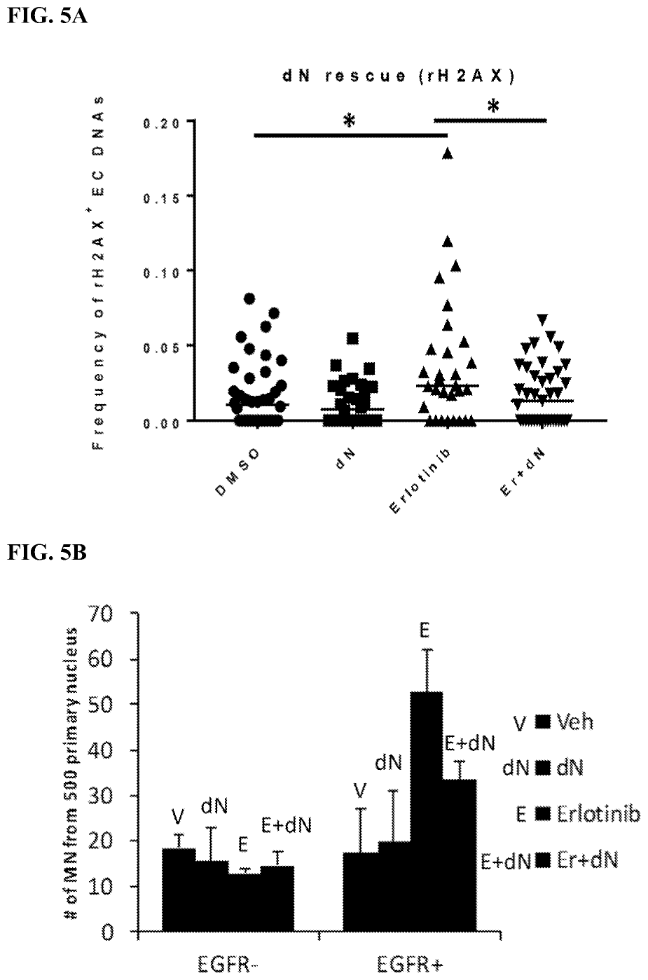

Method of targeting cancers with extrachromosomal (ecDNA) oncogene amplification based on enhanced dependence of ecDNA on de novo nucleotide synthesis. We demonstrate that the addition of de-oxy nucleotides prevents the loss of ecDNA in response to targeted inhibition of the oncogenes contained therein. This dependence of ecDNA on de novo nucleotide synthesis, confirmed biochemically, represents a unique vulnerability generated by extrachromosomal oncogene amplification, which can be targeted by perturbing nucleuotide metabolism. (FIG. 5).

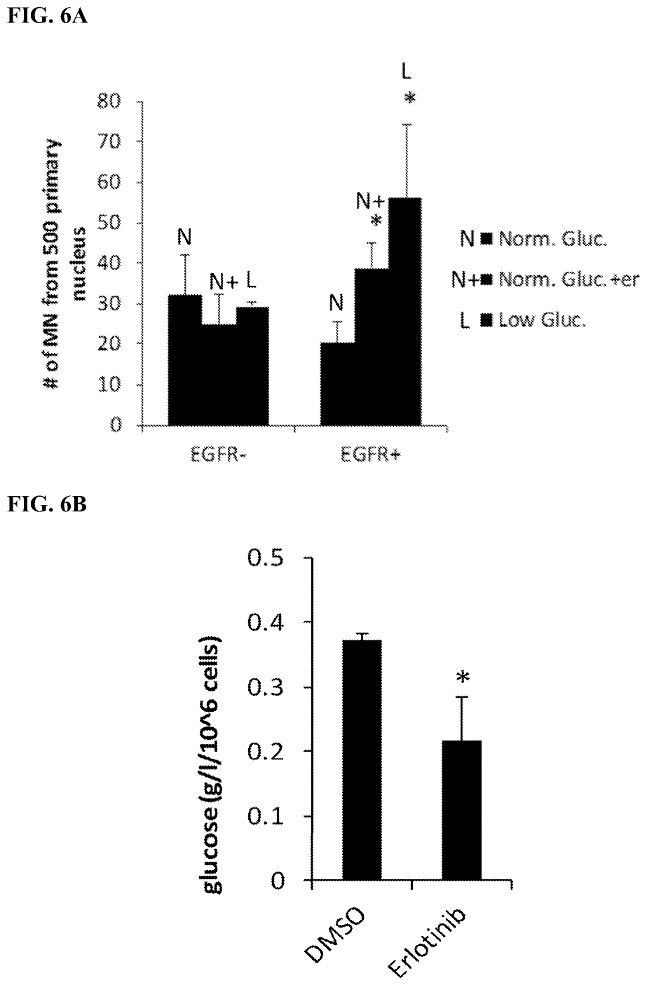

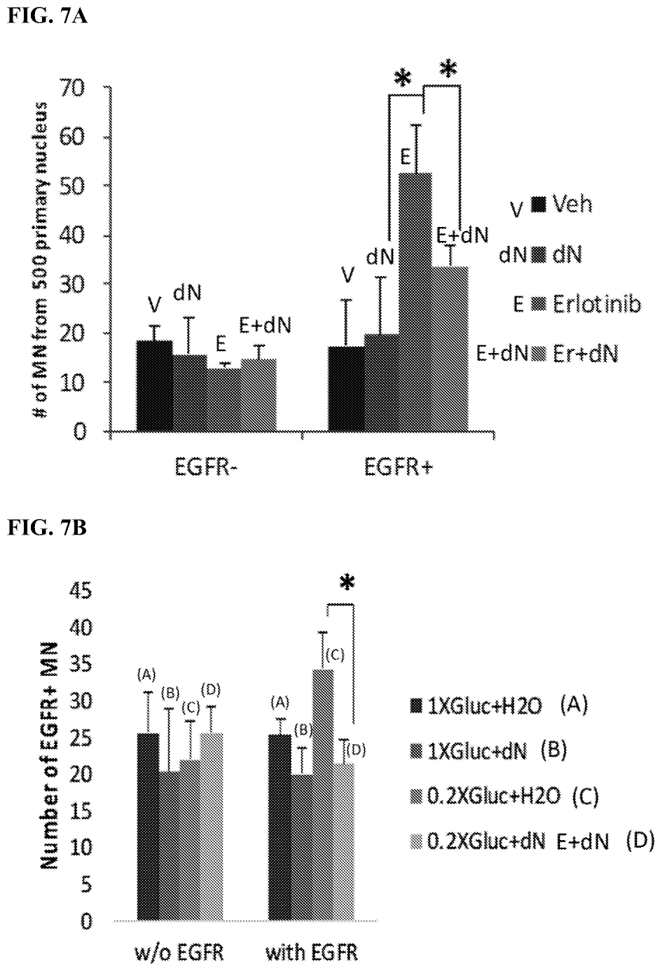

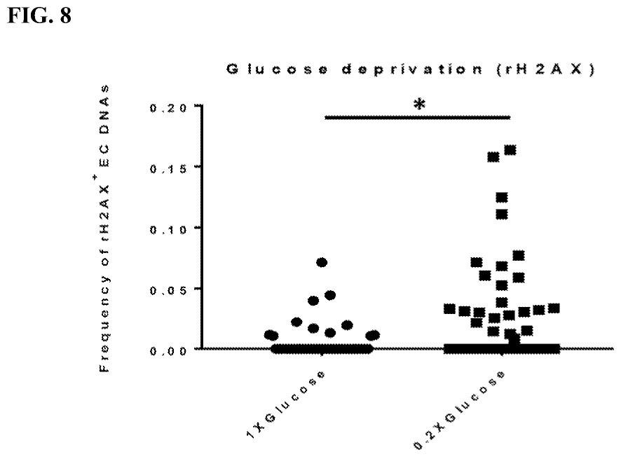

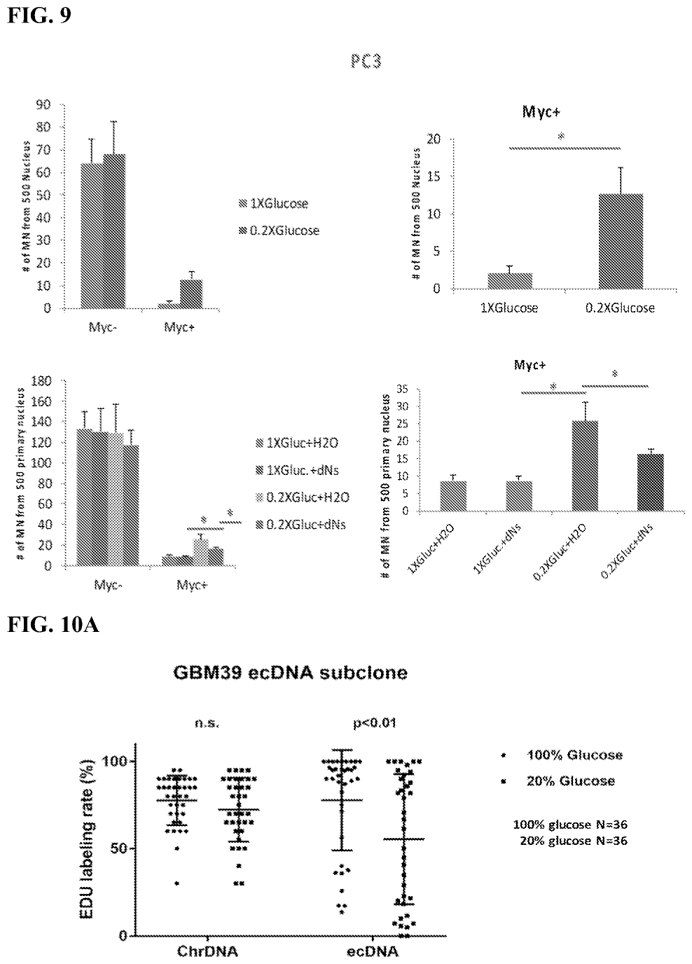

Method of targeting cancers with extrachromosomal (ecDNA) oncogene amplification based on differential metabolic requirements including dependence on glucose: Our lab previously showed that many of the oncogenes that are amplified on ecDNA (Turner et al., Nature, 2017) regulate glucose metabolism (Masui et al., Cell Metabolism, 2013; Babic et al., 2013). We have now found that the targeted inhibitors cause damage to ecDNA by preventing the flux of glucose into cells, thus limiting de novo nucleotide synthesis, resulting in damage specifically to ecDNA, but not chromosomal DNA, leading to micronuclei formation and decreased ecDNA levels and lower oncogene copy number. We find that this is true for multiple cancer types, including GBM and prostate cancer, and multiple oncogenes including EGFR and Myc, which are among the most frequently amplified genes in cancer and which are frequently found on ecDNA (Turner et al., Nature, 2017). FIG. 6-9).

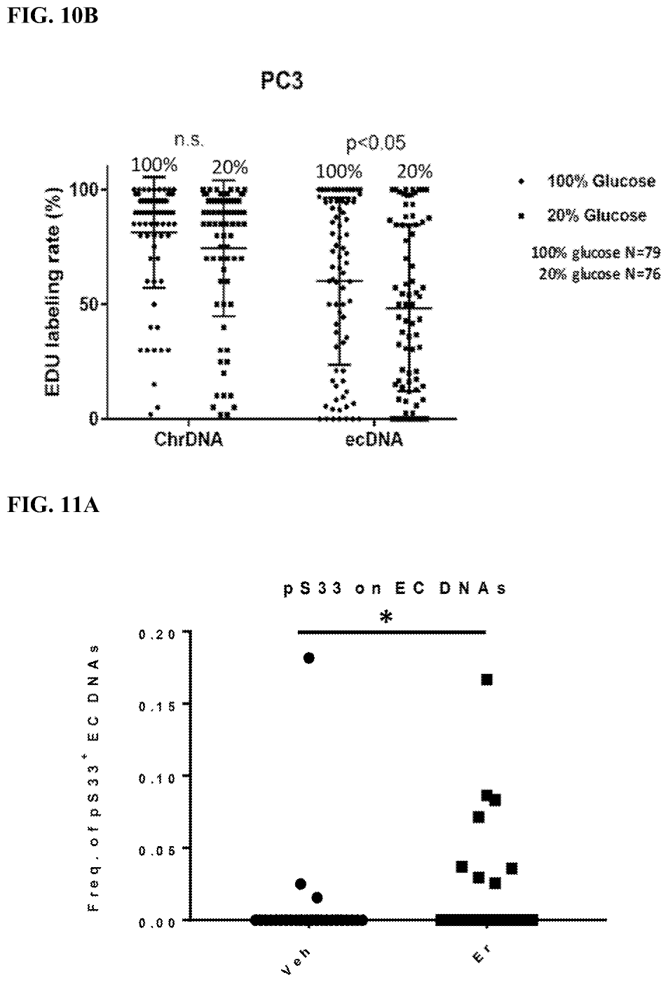

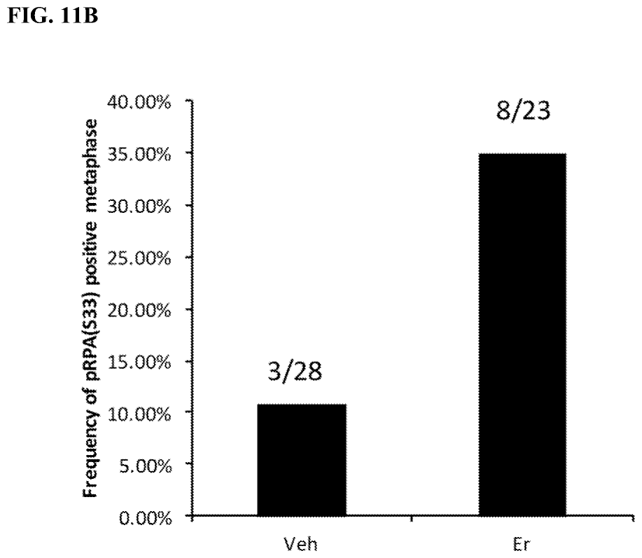

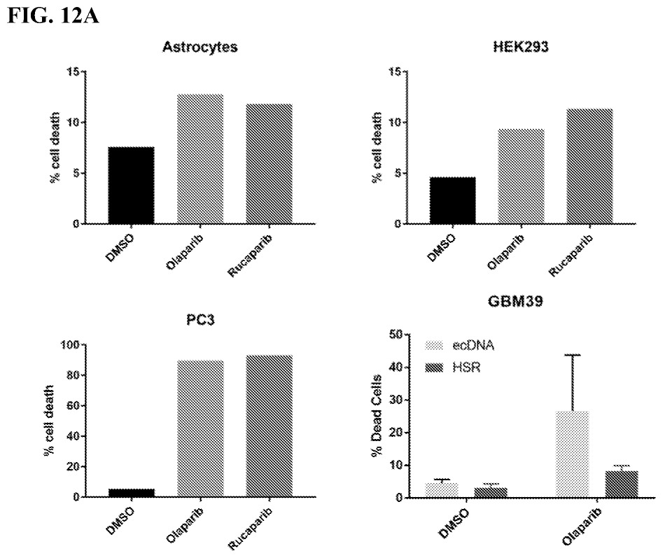

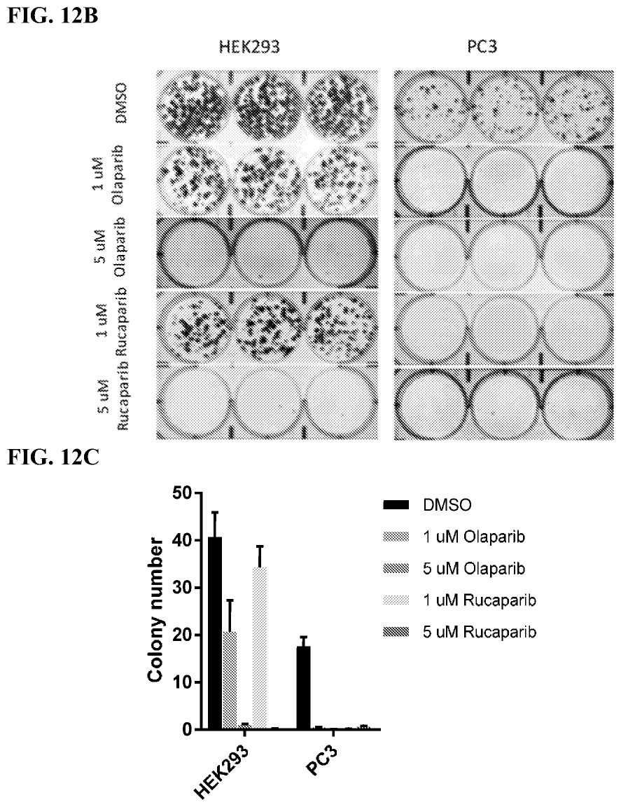

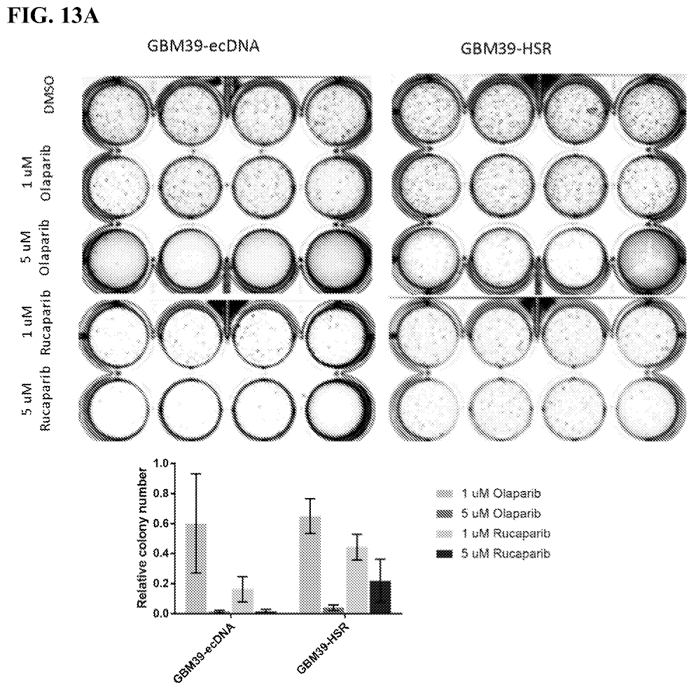

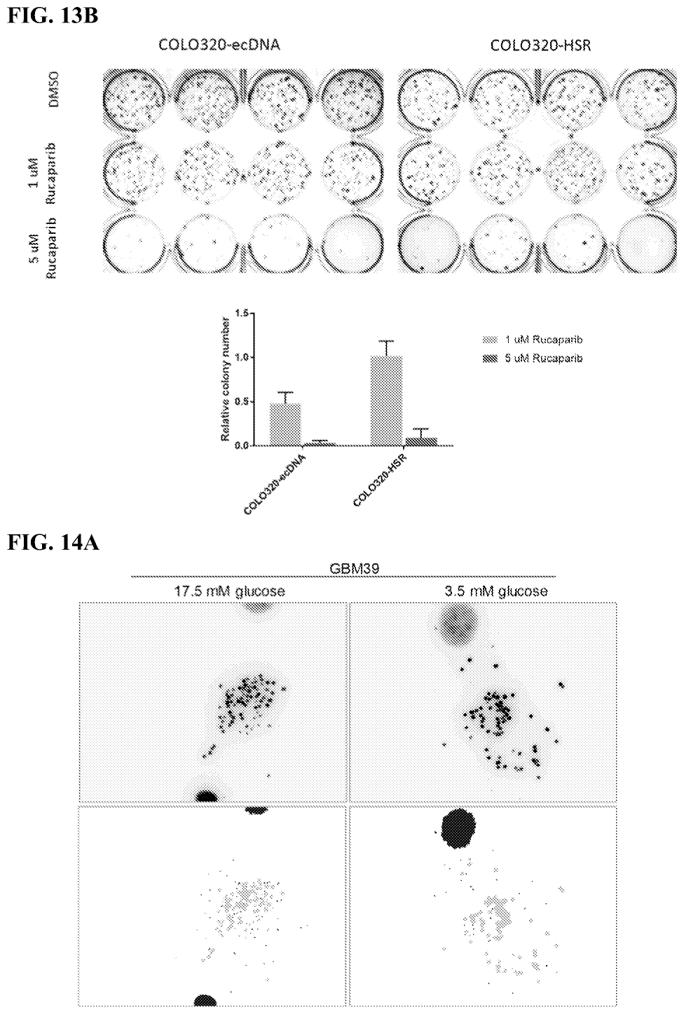

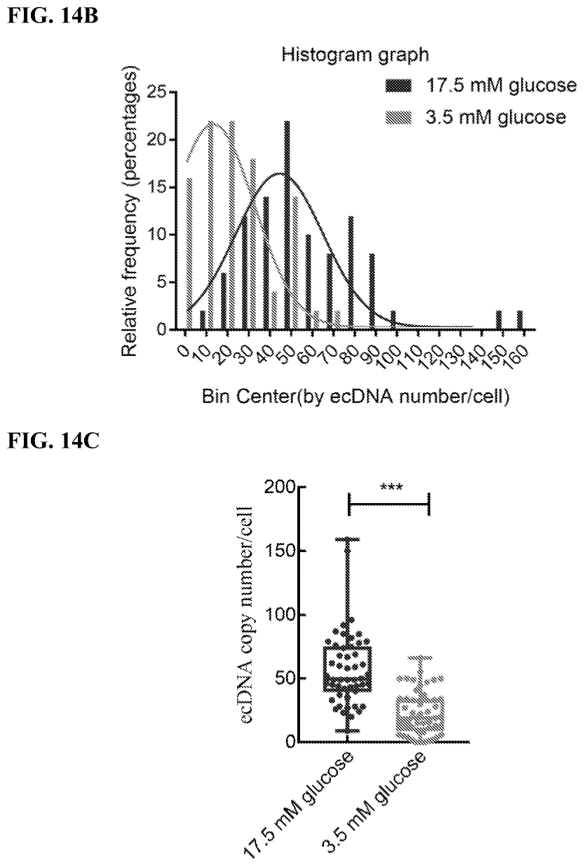

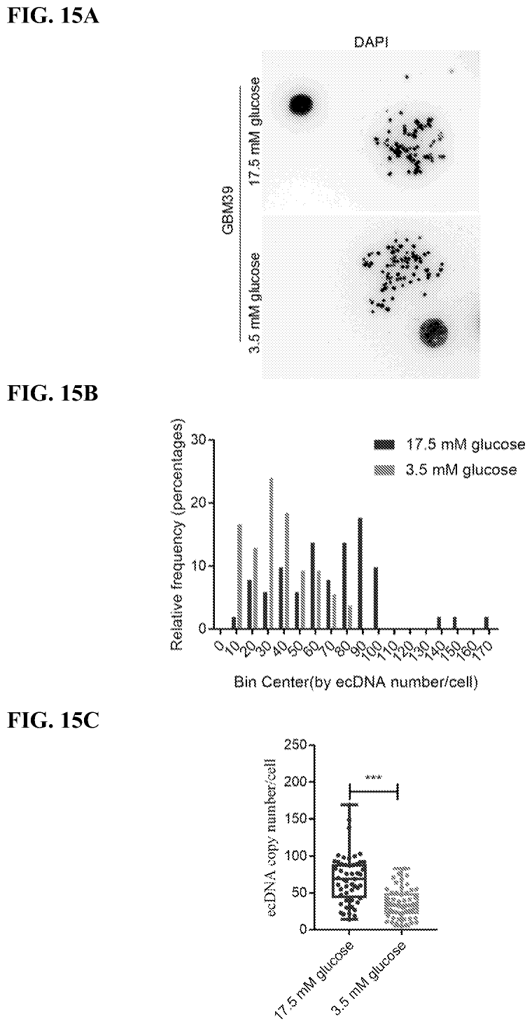

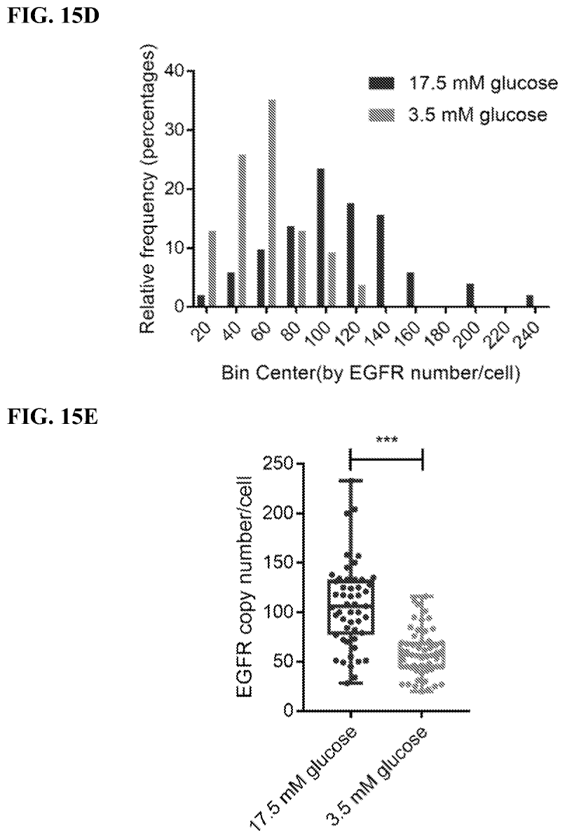

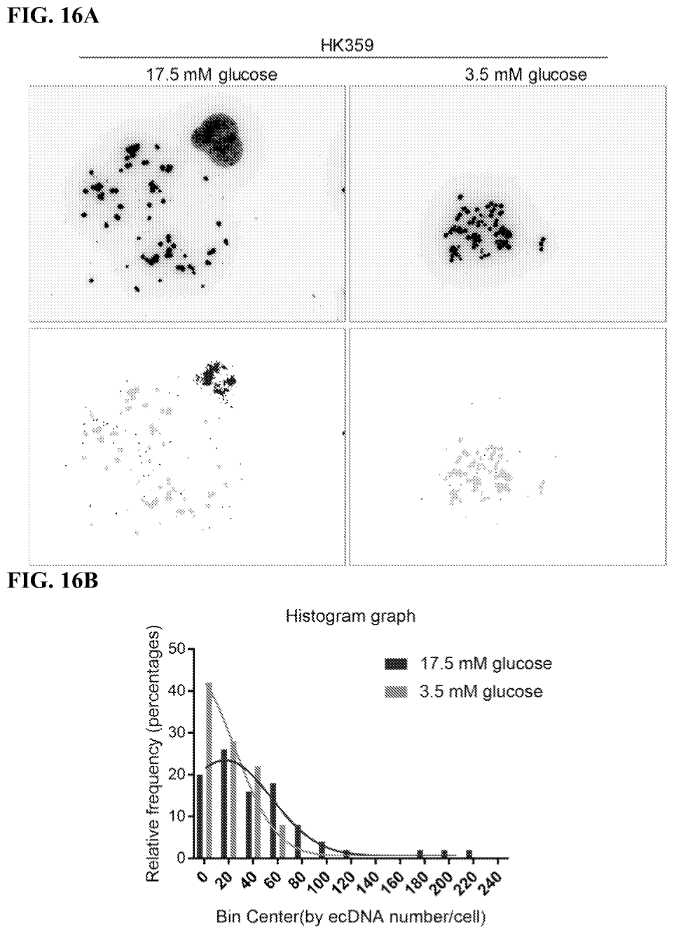

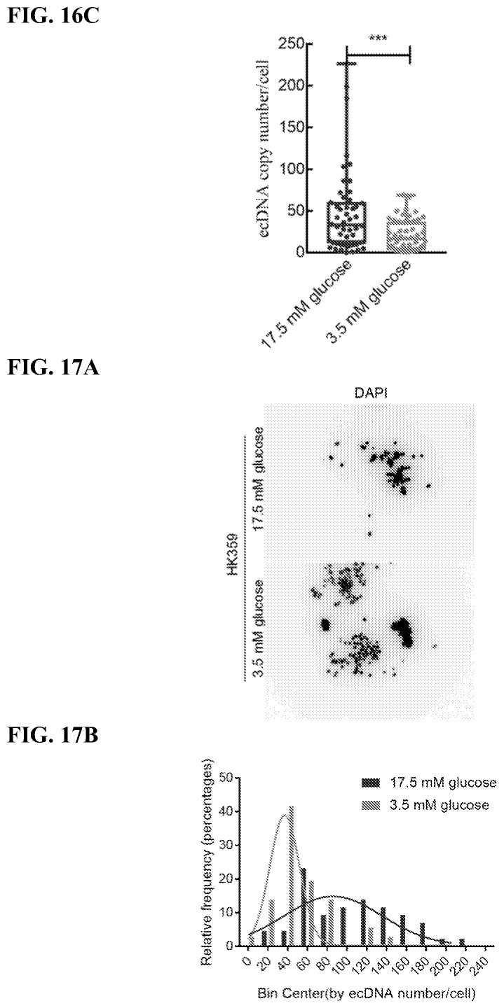

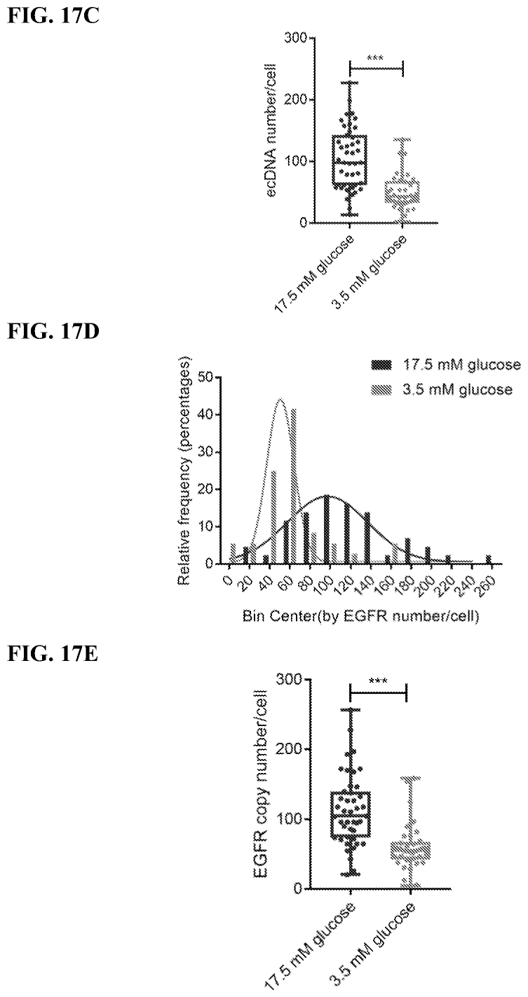

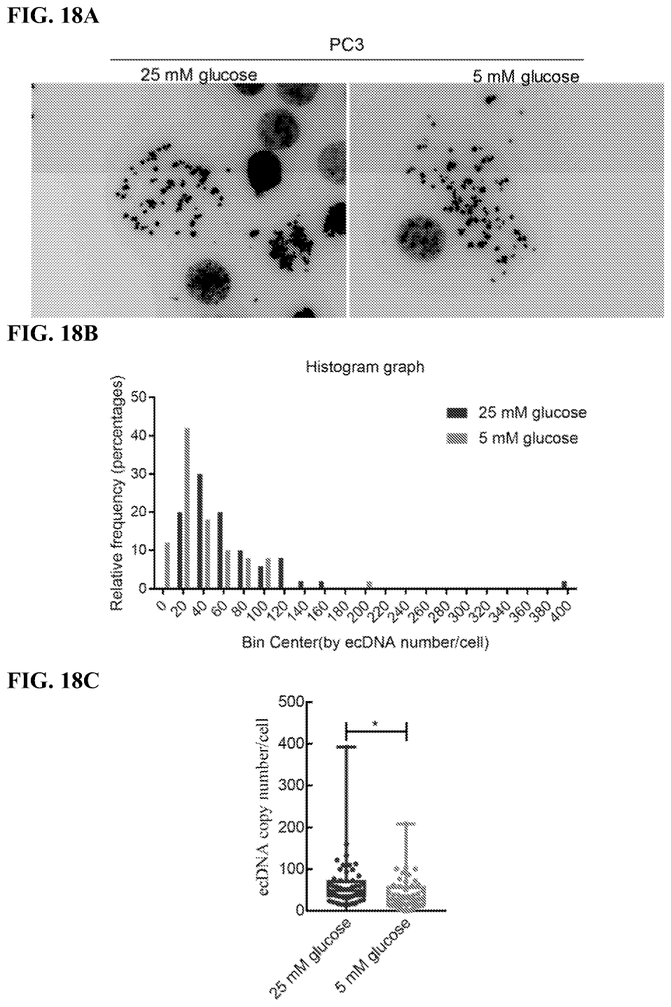

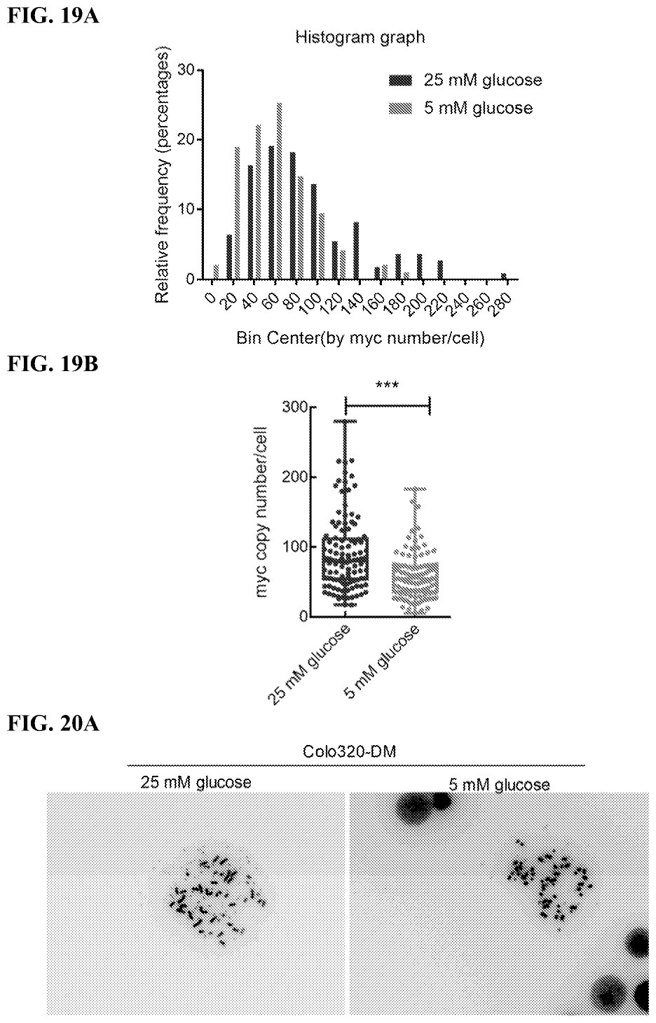

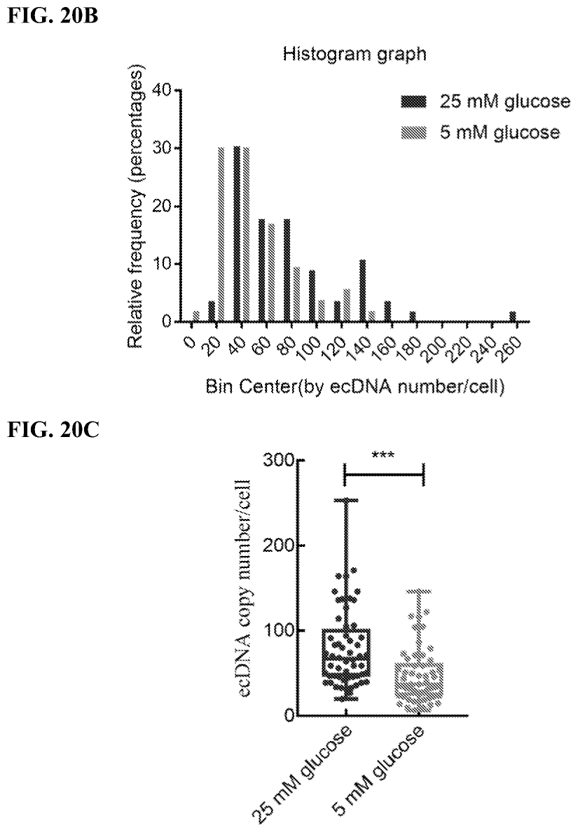

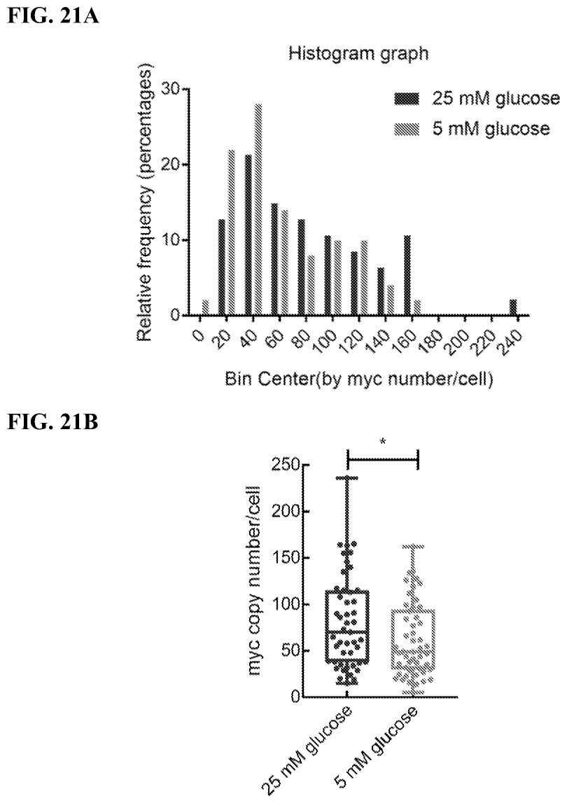

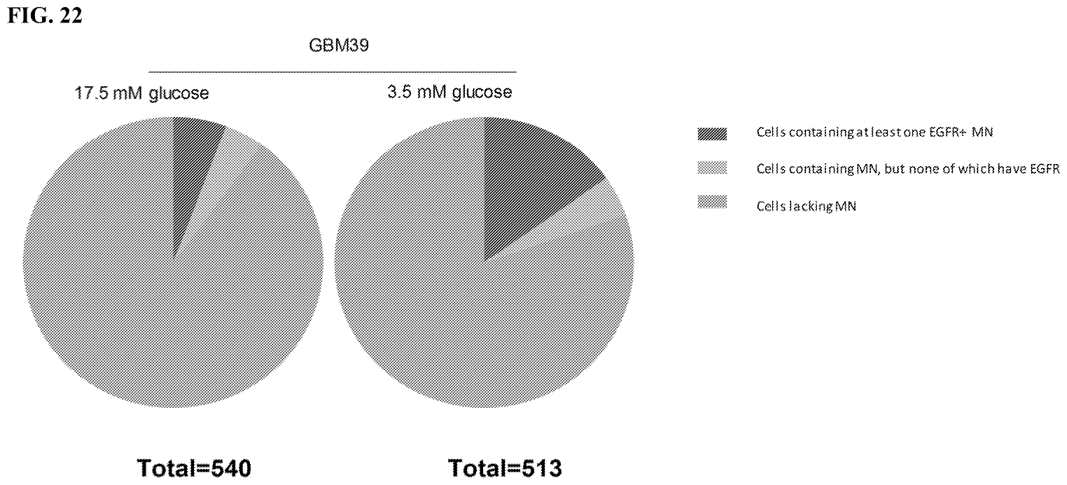

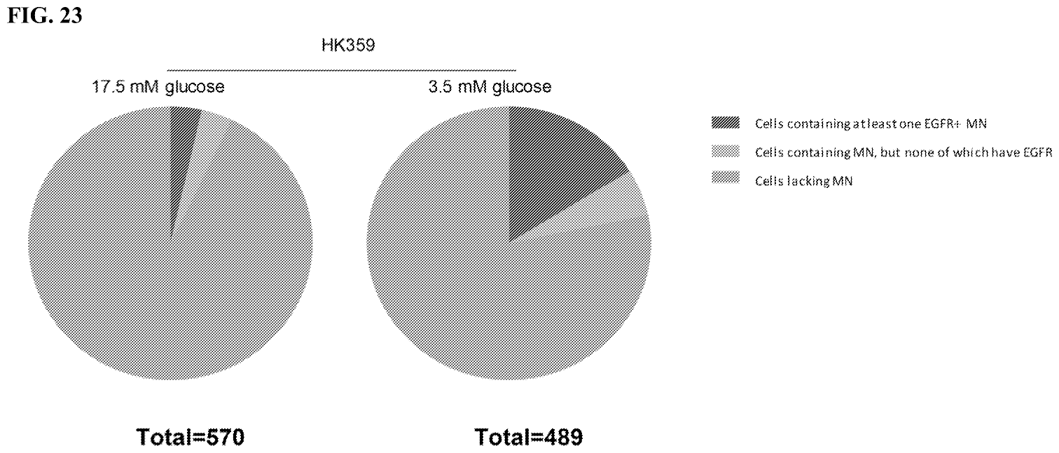

Method of targeting cancers with extrachromosomal (ecDNA) oncogene amplification based on differential DNA replication kinetics and DNA damage and repair mechanisms. Having shown that ecDNA is more dependent on nutrient-mediated de novo nucleotide synthesis, generating a unique vulnerability, we then showed that when glucose levels are limiting, chromosomal DNA continues to replicate, but extrachromosomal DNA replication decreases. These data demonstrate a unique and exploitable metabolic vulnerability of tumor cells with oncogenes amplified on ecDNA. (FIGS. 10-11).

Method of targeting cancers with extrachromosomal (ecDNA) oncogene amplification based on combining approaches provided herein to achieve synergy in targeting.

### Example 2

### Experimental Design

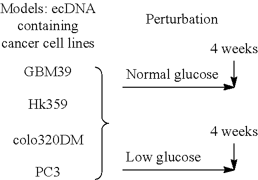

Measurements for Experimental Design: 1) ecDNA number (ecDETECT, Nature, 2017), 2) oncogene number (ecDETECT), 3) histograms to quantify shifts, and 4) micronuclei (MN) number and DNA content.

### REFERENCES

1. Vogelstein, B. et al. Cancer genome landscapes. Science 339, 1546-1558, doi:10.1126/science.1235122 (2013).

2. Stark, G. R., Debatisse, M., Giulotto, E. & Wahl, G. M. Recent progress in understanding mechanisms of mammalian DNA amplification. Cell 57, 901-908 (1989).

3. Schimke, R. T. Gene amplification in cultured animal cells. Cell 37, 705-713 (1984).

4. Fan, Y. et al. Frequency of double minute chromosomes and combined cytogenetic abnormalities and their characteristics. J Appl Genet 52, 53-59, doi:10.1007/s13353-010-0007-z (2011).

5. Nowell, P. C. The clonal evolution of tumor cell populations. Science 194, 23-28 (1976).

6. McGranahan, N. & Swanton, C. Biological and therapeutic impact of intratumor heterogeneity in cancer evolution. Cancer Cell 27, 15-26, doi:10.1016/j.cce11.2014.12.001 (2015).

7. Marusyk, A., Almendro, V. & Polyak, K. Intra-tumour heterogeneity: a looking glass for cancer? Nat Rev Cancer 12, 323-334, doi:10.1038/nrc3261 (2012).

8. Yates, L. R. & Campbell, P. J. Evolution of the cancer genome. Nat Rev Genet 13, 795-806, doi:10.1038/nrg3317 (2012).

9. Greaves, M. & Maley, C. C. Clonal evolution in cancer. Nature 481, 306-313, doi:10.1038/nature10762 (2012).

10. Andor, N. et al. Pan-cancer analysis of the extent and consequences of intratumor heterogeneity. Nat Med 22, 105-113, doi:10.1038/nm.3984 (2016).

11. Gillies, R. J., Verduzco, D. & Gatenby, R. A. Evolutionary dynamics of carcinogenesis and why targeted therapy does not work. Nat Rev Cancer 12, 487-493, doi:10.1038/nrc3298 (2012).

12. Von Hoff, D. D., Needham-VanDevanter, D. R., Yucel, J., Windle, B. E. & Wahl, G. M. Amplified human MYC oncogenes localized to replicating submicroscopic circular DNA molecules. Proc Natl Acad Sci USA 85, 4804-4808 (1988).

13. Garsed, D. W. et al. The architecture and evolution of cancer neochromosomes. Cancer Cell 26, 653-667, doi:10.1016/j.cce11.2014.09.010 (2014).

14. Carroll, S. M. et al. Double minute chromosomes can be produced from precursors derived from a chromosomal deletion. Mol Cell Biol 8, 1525-1533 (1988).

15. Windle, B., Draper, B. W., Yin, Y. X., O'Gorman, S. & Wahl, G. M. A central role for chromosome breakage in gene amplification, deletion formation, and amplicon integration. Genes Dev 5, 160-174 (1991).

16. Kanda, T., Otter, M. & Wahl, G. M. Mitotic segregation of viral and cellular acentric extrachromosomal molecules by chromosome tethering. J Cell Sci 114, 49-58 (2001).

17. Mitelman, F., Johansson, B. & Mertens, F. Mitelman Database of Chromosome Aberrations and Gene Fusions in Cancer, <http://cgap.nci.nih.gov/Chromosomes/Mitelman> (2016).

18. Sanborn, J. Z. et al. Double minute chromosomes in glioblastoma multiforme are revealed by precise reconstruction of oncogenic amplicons. Cancer Res 73, 6036-6045, doi:10.1158/0008-5472.CAN-13-0186 (2013).

19. Almendro, V. et al. Inference of tumor evolution during chemotherapy by computational modeling and in situ analysis of genetic and phenotypic cellular diversity. Cell Rep 6, 514-527, doi:10.1016/j.celrep.2013.12.041 (2014).

20. Zack, T. I. et al. Pan-cancer patterns of somatic copy number alteration. Nat Genet 45, 1134-1140, doi:10.1038/ng.2760 (2013).

21. Nathanson, D. A. et al. Targeted therapy resistance mediated by dynamic regulation of extrachromosomal mutant EGFR DNA. Science 343, 72-76, doi:10.1126/science.1241328 (2014).

22. Storlazzi, C. T. et al. Gene amplification as double minutes or homogeneously staining regions in solid tumors: origin and structure. Genome Res 20, 1198-1206, doi:10.1101/gr.106252.110 (2010).

23. Bozic, I. et al. Accumulation of driver and passenger mutations during tumor progression. Proc Natl Acad Sci USA 107, 18545-18550, doi:10.1073/pnas.1010978107 (2010).

24. Li, X. et al. Temporal and spatial evolution of somatic chromosomal alterations: a case-cohort study of Barrett's esophagus. Cancer Prey Res (Phila) 7, 114-127, doi:10.1158/1940-6207.CAPR-13-0289 (2014).

25. Mishra, S. & Whetstine, J. R. Different Facets of Copy Number Changes: Permanent, Transient, and Adaptive. Mol Cell Biol 36, 1050-1063, doi:10.1128/MCB.00652-15 (2016).

26. Schimke, R. T., Kaufman, R. J., Alt, F. W. & Kellems, R. F. Gene amplification and drug resistance in cultured murine cells. Science 202, 1051-1055 (1978).

27. Nikolaev, S. et al. Extrachromosomal driver mutations in glioblastoma and low-grade glioma. Nat Commun 5, 5690, doi:10.1038/ncomms6690 (2014).

28. Biedler, J. L., Schrecker, A. W. & Hutchison, D. J. Selection of chromosomal variant in amethopterin-resistant sublines of leukemia L1210 with increased levels of dihydrofolate reductase. J Natl Cancer Inst 31, 575-601 (1963).

29. Lee, P. M. Bayesian statistics: an introduction. 4th edn, (John Wiley & Sons, 2012).

30. Motl, J. <https://www.mathworks.com/matlabcentral/fileexchange/40854>

31. Bradley, D. & Roth, G. Adaptive thresholding using the integral image. Journal of graphics, gpu, and game tools 12, 13-21 (2007).

32. Lander, E. S. et al. Initial sequencing and analysis of the human genome. Nature 409, 860-921, doi:10.1038/35057062 (2001).

33. Kent, W. J. et al. The human genome browser at UCSC. Genome Res 12, 996-1006, doi:10.1101/gr.229102. Article published online before print in May 2002 (2002).

34. Li, H. & Durbin, R. Fast and accurate short read alignment with Burrows-Wheeler transform. Bioinformatics 25, 1754-1760, doi:10.1093/bioinformatics/btp324 (2009).

35. Miller, C. A., Hampton, O., Coarfa, C. & Milosavljevic, A. ReadDepth: a parallel R package for detecting copy number alterations from short sequencing reads. PLoS One 6, e16327, doi:10.1371/journal.pone.0016327 (2011).

36. Pavlova, N. N. & Thompson, C. B. The Emerging Hallmarks of Cancer Metabolism. Cell Metab 23, 27-47, doi:10.1016/j.cmet.2015.12.006 (2016).

37. Tuzun, E. et al., Nat. Genet. 37, 727 (2005).

38. Eichler, E. E. et al., Nature 447, 161 (2007).

39. Derrien, T. et al., PLoS ONE 7, e30377 (2012).

40. Rosenbloom, K. R. et al., Nucleic Acids Res. 43, D670 (2015).

41. Bailey, J. A., Yavor, A. M., Massa, H. F., Trask, B. J., Eichler, E. E., Genome Res. 11, 1005 (2001).

42. Bailey, J. A. et al., Science 297, 1003 (2002).

43. Comaniciu, D. and Meer, P., IEEE Transactions on pattern analysis and machine intelligence 24, 603 (2002).

44. Abyzov, A., Urban, A. E., Snyder, M., Gerstein, M., Genome Res. 21, 974 (2011).

45. The Cancer Genome Atlas (TCGA) Research Network, Nature 455, 1061 (2008).

46. Forbes, S. A. et al., Nucleic Acids Res. 43, D805 (2015).

47. Bozic, I. et al., Proc. Natl. Acad. Sci. U.S.A. 107, 18545 (2010).

48. Pavlova, N. N., Thompson, C. B., Cell Metab. 23, 27 (2016).

49. DeVita, V. T., Young, R. C., Canellos, G. P., Cancer 35, 98 (1975).

50. Del Monte, U., Cell Cycle 8, 505 (2009).

51. Dempsey, M. F., Condon, B. R., Hadley, D. M., AJNR Am J Neuroradiol 26, 770 (2005).

52. Waclaw, B. et al., Nature 525, 261 (2015).

53. Li, X., et al., Cancer Prey Res (Phila) 7, 114 (2014).

Turner et al., Nature, 2017 Mar. 2; 543(7643): 122-125. doi: 10.1038/nature21356.

Masui et al., Cell Metabolism, 2013 Nov. 5, Vol. 18, Issue 5, p. 726-739.

Babic et al., Cell Metabolism, 2013 Jun. 4, Vol. 17, Issue 6, p. 1000-1008.

### P Embodiments

Embodiment P1. A method of treating cancer in a subject in need thereof, wherein the cancer amplifies an extrachromosomal (ecDNA) oncogene, the method including administering a therapeutically effective amount of a targeted agent capable of lowering the DNA copy number of the extrachromosomal (ecDNA) oncogene.

Embodiment P2. The method of embodiment P1 wherein the targeted agent is capable of decreasing de novo nucleotide synthesis increased within the cancer relative to a non-cancer cell.

Embodiment P3. The method of embodiment P1 wherein the targeted agent is capable of decreasing a metabolic process increased within the cancer relative to a non-cancer cell (e.g. a glucose-dependent metabolic process).

Embodiment P4. The method of embodiment P1 wherein the targeted agent is capable of decreasing a DNA replication kinetic parameter, DNA damage parameter and/or DNA repair parameter increased within the cancer relative to a non-cancer cell.

### Embodiments

Embodiment 1. A method of treating cancer in a human subject having or being at risk of developing cancer, said method comprising administering to said human subject an effective amount of a DNA repair pathway inhibitor, thereby treating cancer in said subject, wherein said human subject has been identified as having an amplified extrachromosomal oncogene.

Embodiment 2. The method of embodiment 1, said method comprising prior to said administering, detecting an amplified extrachromosomal oncogene in a cancer cell in a first biological sample obtained from said human subject by contacting said biological sample with an oncogene-binding agent and detecting binding of said oncogene-binding agent to said amplified extrachromosomal oncogene.

Embodiment 3. A method of treating cancer in a human subject in need thereof, said method comprising:
(i) detecting an amplified extrachromosomal oncogene in a cancer cell in a first biological sample obtained from a human subject having or being at risk of developing cancer by contacting said biological sample with an oncogene-binding agent and detecting binding of said oncogene-binding agent to said amplified extrachromosomal oncogene; and
(ii) administering to said human subject an effective amount of a DNA repair pathway inhibitor thereby treating cancer in said subject.

Embodiment 4. The method of any one of embodiments 1-3, wherein said amplified extrachromosomal oncogene forms part of a circular extrachromosomal DNA.

Embodiment 5. The method of any one of embodiments 2-4, wherein said detecting comprises detecting a level of said circular extrachromosomal DNA relative to a standard control.

Embodiment 6. The method of any one of embodiments 2-5, wherein said detecting comprises mapping said circular extrachromosomal DNA.

Embodiment 7. The method of any one of embodiments 2-6, wherein said detecting comprises detecting genetic heterogeneity of said circular extrachromosomal DNA relative to a standard control.

Embodiment 8. The method of any one of embodiments 2-7, wherein said oncogene-binding agent is a nucleic acid, a peptide nucleic acid or a protein.

Embodiment 9. The method of any one of embodiments 2-8, wherein said oncogene-binding agent is a labeled nucleic acid, a labeled peptide nucleic acid or a labeled protein.

Embodiment 10. The method of any one of embodiments 1-8, wherein said amplified extrachromosomal oncogene is EGFR, c-Myc, N-Myc, cyclin D1, ErbB2, CDK4, CDK6, BRAF, MDM2, or MDM4.

Embodiment 11. The method of any one of embodiments 2-10, wherein said first biological sample is a blood-derived sample, a urine-derived sample, a tumor sample, or a tumor fluid sample.

Embodiment 12. The method of any one of embodiments 1-11, wherein said DNA repair pathway inhibitor is a peptide, small molecule, nucleic acid, antibody or aptamer.

Embodiment 13. The method of any one of embodiments 1-12, wherein said DNA repair pathway inhibitor is a poly ADP ribose polymerase (PARP) inhibitor.

Embodiment 14. The method of any one of embodiments 1-13, wherein said DNA repair pathway inhibitor is rucaparib or olaparib.

Embodiment 15. The method of any one of embodiments 1-14, wherein said cancer is sarcoma, glioblastoma, lung cancer, esophageal cancer, breast cancer, bladder cancer or stomach cancer.

Embodiment 16. The method of any one of embodiments 2-15, wherein said detecting comprises detecting a first level of said amplified extrachromosomal oncogene.

Embodiment 17. The method of embodiment 16, comprising after step (ii):
(iii) obtaining a second biological sample from said subject;
(iv) detecting a second level of said amplified extrachromosomal oncogene; and
(v) comparing said first level to said second level.

Embodiment 18. The method of embodiment 17, wherein said first biological sample is obtained at a time t0, from said subject and said second biological sample is obtained at a later time t1 from said subject.

Embodiment 19. The method of embodiment 18, wherein said first level of said amplified extrachromosomal oncogene is a first amount of oncogene copies or fragments thereof and said second level of said amplified extrachromosomal oncogene is a second amount of oncogene copies or fragments thereof.

Embodiment 20. A method of treating cancer in a human subject in need thereof, said method comprising:
(i) detecting a first level of an amplified extrachromosomal oncogene in a cancer cell in a first biological sample obtained from a human subject having or being at risk of developing cancer;
(ii) administering to said human subject an effective amount of a DNA repair pathway inhibitor;
(iii) detecting a second level of an amplified extrachromosomal oncogene in a cancer cell in a second biological sample obtained from said human subject; and
(iv) comparing said first level to said second level, thereby treating cancer in said human subject.

Embodiment 21. The method of embodiment 20, wherein said detecting in step (i) and (iii) comprises contacting said first and second biological sample with an oncogene-binding agent and detecting binding of said oncogene-binding agent to said amplified extrachromosomal oncogene.

Embodiment 22. The method of embodiment 21, wherein said oncogene-binding agent is a labeled nucleic acid probe.

Embodiment 23. The method of any one of embodiments 20-22, wherein said amplified extrachromosomal oncogene is EGFR, c-Myc, N-Myc, cyclin D1, ErbB2, CDK4, CDK6, BRAF, MDM2, or MDM4.

Embodiment 24. The method of any one of embodiments 20-23, wherein said first or second biological sample is a blood-derived sample, a urine-derived sample, a tumor sample, or a tumor fluid sample.

Embodiment 25. The method of any one of embodiments 20-24, wherein said DNA repair pathway inhibitor is a peptide, small molecule, nucleic acid, antibody or aptamer.

Embodiment 26. The method of any one of embodiments 20-25, wherein said DNA repair pathway inhibitor is a poly ADP ribose polymerase (PARP) inhibitor.

Embodiment 27. The method of any one of embodiments 20-26, wherein said DNA repair pathway inhibitor is rucaparib or olaparib.

Embodiment 28. The method of any one of embodiments 20-27, wherein said cancer is sarcoma, glioblastoma, lung cancer, esophageal cancer, breast cancer, bladder cancer or stomach cancer.

1. A method of treating cancer in a human subject having or being at risk of developing cancer, said method comprising administering to said human subject an effective amount of a DNA repair pathway inhibitor, thereby treating cancer in said subject, wherein said human subject has been identified as having an amplified extrachromosomal oncogene.

2. The method of claim 1, said method comprising prior to said administering, detecting an amplified extrachromosomal oncogene in a cancer cell in a first biological sample obtained from said human subject by contacting said biological sample with an oncogene-binding agent and detecting binding of said oncogene-binding agent to said amplified extrachromosomal oncogene.

3. A method of treating cancer in a human subject in need thereof, said method comprising:
(i) detecting an amplified extrachromosomal oncogene in a cancer cell in a first biological sample obtained from a human subject having or being at risk of developing cancer by contacting said biological sample with an oncogene-binding agent and detecting binding of said oncogene-binding agent to said amplified extrachromosomal oncogene; and
(ii) administering to said human subject an effective amount of a DNA repair pathway inhibitor thereby treating cancer in said subject.

4. The method of claim 1, wherein said amplified extrachromosomal oncogene forms part of a circular extrachromosomal DNA.

5. The method of claim 2, wherein said detecting comprises detecting a level of said circular extrachromosomal DNA relative to a standard control.

6. The method of claim 2, wherein said detecting comprises mapping said circular extrachromosomal DNA.

7. The method of claim 2, wherein said detecting comprises detecting genetic heterogeneity of said circular extrachromosomal DNA relative to a standard control.

8. The method of claim 2, wherein said oncogene-binding agent is a nucleic acid, a peptide nucleic acid or a protein.

9. The method of claim 2, wherein said oncogene-binding agent is a labeled nucleic acid, a labeled peptide nucleic acid or a labeled protein.

10. The method of claim 1, wherein said amplified extrachromosomal oncogene is EGFR, c-Myc, N-Myc, cyclin D1, ErbB2, CDK4, CDK6, BRAF, MDM2, or MDM4.

11. The method of claim 2, wherein said first biological sample is a blood-derived sample, a urine-derived sample, a tumor sample, or a tumor fluid sample.

12. The method of claim 1, wherein said DNA repair pathway inhibitor is a peptide, small molecule, nucleic acid, antibody or aptamer.

13. The method of claim 1, wherein said DNA repair pathway inhibitor is a poly ADP ribose polymerase (PARP) inhibitor.

14. The method o of claim 1, wherein said DNA repair pathway inhibitor is rucaparib or olaparib.

15. The method of claim 1, wherein said cancer is sarcoma, glioblastoma, lung cancer, esophageal cancer, breast cancer, bladder cancer or stomach cancer.

16. The method of claim 2, wherein said detecting comprises detecting a first level of said amplified extrachromosomal oncogene.

17. The method of claim 16, comprising after step (ii):
(iii) obtaining a second biological sample from said subject;
(iv) detecting a second level of said amplified extrachromosomal oncogene; and
(v) comparing said first level to said second level.

18. The method of claim 17, wherein said first biological sample is obtained at a time t₀, from said subject and said second biological sample is obtained at a later time t₁ from said subject.

19. The method of claim 18, wherein said first level of said amplified extrachromosomal oncogene is a first amount of oncogene copies or fragments thereof and said second level of said amplified extrachromosomal oncogene is a second amount of oncogene copies or fragments thereof.

20. A method of treating cancer in a human subject in need thereof, said method comprising:
(i) detecting a first level of an amplified extrachromosomal oncogene in a cancer cell in a first biological sample obtained from a human subject having or being at risk of developing cancer;
(ii) administering to said human subject an effective amount of a DNA repair pathway inhibitor;
(iii) detecting a second level of an amplified extrachromosomal oncogene in a cancer cell in a second biological sample obtained from said human subject; and
(iv) comparing said first level to said second level, thereby treating cancer in said human subject.

21. The method of claim 20, wherein said detecting in step (i) and (iii) comprises contacting said first and second biological sample with an oncogene-binding agent and detecting binding of said oncogene-binding agent to said amplified extrachromosomal oncogene.

22. The method of claim 21, wherein said oncogene-binding agent is a labeled nucleic acid probe.

23. The method of claim 20, wherein said amplified extrachromosomal oncogene is EGFR, c-Myc, N-Myc, cyclin D1, ErbB2, CDK4, CDK6, BRAF, MDM2, or MDM4.

24. The method of claim 20, wherein said first or second biological sample is a blood-derived sample, a urine-derived sample, a tumor sample, or a tumor fluid sample.

25. The method of claim 20, wherein said DNA repair pathway inhibitor is a peptide, small molecule, nucleic acid, antibody or aptamer.

26. The method o of claim 20, wherein said DNA repair pathway inhibitor is a poly ADP ribose polymerase (PARP) inhibitor.

27. The method of claim 20, wherein said DNA repair pathway inhibitor is rucaparib or olaparib.

28. The method of claim 20, wherein said cancer is sarcoma, glioblastoma, lung cancer, esophageal cancer, breast cancer, bladder cancer or stomach cancer.
